(19) United States
(12) Patent Application Publication
(10) Pub. No.: US 2025/0223249 A1
(43) Pub. Date: Jul. 10, 2025

(54) METHOD FOR THE PREPARATION OF A VOLATILE MIXTURE OF ALKANES AND A COSMETIC COMPOSITION

(71) Applicant: BIOSYNTHIS, Saint Cyr Sous Dourdan (FR)

(72) Inventor: Thierry BERNOUD, Saint Cyr Sous Dourdan (FR)

(73) Assignee: BIOSYNTHIS, Saint Cyr Sous Dourdan (FR)

(21) Appl. No.: 18/853,474

(22) PCT Filed: Apr. 3, 2023
(86) PCT No.: PCT/EP2023/058723

§ 371 Date: Dec. 3, 2024

(30) Foreign Application Priority Data
Apr. 2, 2022 (FR) FR2203025

(51) Int. Cl. C07C 29/34; A61K 8/31; A61Q 1/06; A61Q 1/10; A61Q 5/00; A61Q 19/00; C07C 1/24; C07C 5/03

(52) U.S. Cl. CPC C07C 29/34; A61K 8/31; A61Q 1/06; A61Q 1/10; A61Q 5/00; A61Q 19/00; C07C 1/24; C07C 5/03; A61K 2800/5922

(57) ABSTRACT

A method for the preparation of a cosmetic composition includes a step of mixing volatile mixture of alkanes obtained by a method for preparing a volatile mixture of alkanes including: 1) producing a Guerbet alcohol by a Guerbet synthesis from a biosourced linear or branched alcohol with a mass purity greater than or equal to 80%; 2) adding to the Guerbet alcohol at least one biosourced linear or branched alcohol; and 3) obtaining a volatile mixture of alkanes made of the branched alkane from Guerbet alcohol and at least one alkane from the at least one biosourced alcohol added in step 2 by a dehydration reaction followed by a hydrogenation reaction of the mixture of biosourced alcohols formed in step 2, with at least one compound selected from among non-volatile oils, glossy oils, pasty fatty substances, gelling agents, film-forming polymers, waxes, antioxidants, pigments, surfactants, an aqueous phase and mixtures thereof.

The present application relates to a process for preparing a volatile mixture of alkanes.

The present application also relates to a process for preparing a cosmetic composition comprising this volatile mixture of alkanes.

The invention also relates to the resulting cosmetic composition.

As part of this request, a number of definitions must be given.

An “alkane” is a saturated hydrocarbon consisting only of carbon and hydrogen atoms linked together by simple covalent bonds whose general formula is CₙH₂ₙ₊₂.

A “straight alkane” is an alkane in which each carbon atom is bonded to a maximum of two carbon atoms.

A “branched alkane” is an alkane in which some carbon atoms are linked to three or even four carbon atoms.

A “Cₓ alkane” is an alkane consisting of x carbon atoms, and the alkane may be linear or branched. For example, dodecane is a C₁₂ alkane.

A “C>x alkane” is an alkane consisting of at least x+1 carbon atoms, and the alkane may be linear or branched. For example, dodecane is a C>10 linear alkane or C₁₂ alkane.

A “C≤x alkane” is an alkane consisting of at most x carbon atoms or less than x carbon atoms, said alkane being able to be linear or branched. For example, 2,3,6-trimethylheptane (CAS 4032-93-3) is a C≤10 alkane.

A “Cₓ-C_{y}” alkane is an alkane consisting of x to y carbon atoms, and the alkane may be linear or branched. For example, “C₁-C₂” alkanes are methane and ethane.

Isoalkane is a branched alkane containing at least one carbon atom linked to at least three carbon atoms including a methyl group.

An “alcohol” is an organic compound in which one of the carbon atoms (this being tetrahedral) is linked to a group hydroxyl (—OH).

A “primary alcohol” is an alcohol whose carbon containing the hydroxyl group is linked to at least two hydrogen atoms and an organic radical R: R—CH₂—OH.

A «secondary alcohol» is an alcohol whose carbon containing the hydroxyl group is linked to one hydrogen atom and two organic radicals R and R′: R(R′)—CH—OH.

A “Cₓ alcohol” is an alcohol consisting of x carbon atoms, said alcohol may be linear or branched, primary or secondary. For example, n-dodecanol (CAS 123-51-3) is a C₁₂ alcohol.

A “C≤x alcohol” is an alcohol consisting of at most x carbon atoms or less than x carbon atoms, said alcohol being able to be linear or branched, primary or secondary. For example, isoamyl alcohol (CAS 123-51-3) is a C≤10 alcohol.

A “C>10 alcohol” is an alcohol consisting of at least x+1 carbon atoms, said alcohol being able to be linear or branched, primary or secondary. For example, n-dodecanol (CAS 123-51-3) is a C>10 alcohol, or a C₁₂ alcohol.

Commonly, a “volatile mixture of alkanes” is a mixture of alkanes having a flash point less than or equal to 95° C. measured according to the ATSM D93 standard.

For the purposes of the present invention, the term “volatile mixture of alkanes” means a mixture of alkanes having a flash point less than or equal to 55° C. measured according to the ATSM D93 standard.

The flash point is the flash point measured according to ASTM D93. The unit of the flash point measured according to ASTM D93 is degrees Celsius (° C.). It can also be an average of values, each of the values being obtained by means of a measurement according to ASTM D93.

The term “mass percentage” refers to the ratio of the mass of a first compound to the total mass of a mixture of compounds or composition, reduced to a percentage. For example, if 10 grams of a compound y are present in a mixture z having a total mass of 100 grams, then the mass percentage of y in z is 10%.

The term “biosourced” refers to a compound or an organic composition defined as one in which the organic carbon present in the compound or composition is 100% of plant origin, i.e. the ratios of ¹⁴C/¹²C or ¹⁴C/¹³ determined by radiocarbon analysis according to one of the following standards: ASTM D6866, EN 16640 or EN 16785-1 correspond to the ratios of living plant products.

For the purposes of the present invention, the OECD 301F method makes it possible to assess the biodegradability of a substance by a manometric respirometry test.

The Guerbet synthesis is an organic condensation reaction, in which a primary or secondary alcohol bearing a methylene group adjacent to the carbon bearing the alcohol function condenses with itself or with another alcohol, with the release of water. This reaction leads to what are commonly called Guerbet alcohols.

For the purposes of the present invention, the term “Guerbet alcohol” refers to the product obtained by a Guerbet synthesis resulting from the condensation or dimerization with itself of an alcohol compound. In the context of the present invention, the Guerbet synthesis is carried out from a single alcohol compound.

The use of volatile silicone oils in cosmetic products is set to disappear, in particular the use of cyclomethicones which are short-chain cyclic silicone compounds of formula [Si(CH₃)₂—O]ₙ with n between 3 and 7 (limits included). Indeed, their potential harmful effects on the environment, and even on human health, have recently been highlighted.

Research was then carried out to identify compounds likely to offer a satisfactory alternative, these compounds having to have similar volatility characteristics and provide similar properties to the formulation, in terms of viscosity and sensory properties in particular.

This research has highlighted the potential of alkane mixtures to reduce the use of volatile silicones in cosmetic products, or even potentially eliminate them. The alkane mixtures of interest can be complex, include several compounds and several types of alkanes, linear alkanes and branched alkanes. For example, patent application WO2009/064790A1 uses a mixture of three alkane fractions: a C₁₂-C₁₄ isoalkane fraction, a C₁₃-C₁₅ isoalkane fraction and a so-called C₁₃-C₁₅ alkane mixture fraction in very specific proportions. Obtaining properties at least similar to those obtained using volatile silicones is a real challenge and requires fine optimization of the composition of the mixture both in terms of proportions and structures of the alkanes used.

To date, the alkanes used as volatile solvents in cosmetic formulations, mainly isododecane, are mainly derived from petrochemicals. However, with limited oil resources and a fluctuating and polluting oil market, meeting the demand for volatile alkanes for cosmetic formulations represents a strategic challenge for the cosmetics industry.

Branched alkanes are generally obtained as mixtures by polymerization or copolymerization reactions of unsaturated monomers, isobutene or isobutene/butene mixtures. Linear C>6 alkanes come from the cracking of heavy alkanes, a process leading in particular to the formation of unsaturated by-products of the alkene type.

The processes for producing alkanes from petrochemicals require the development of complex and costly purification processes due to the multiple by-products. Indeed, even in trace amounts, these impurities can have a significant impact on the quality of the alkanes obtained and significantly limit their use.

The presence of alkene-type impurities has a deleterious effect on the storage stability of cosmetic formulations. Indeed, the oxidation of unsaturated bonds in the presence of oxygen leads in particular to accelerated degradation of the components of the composition.

In addition, alkenes and degradation products appearing during storage have an unpleasant odor that must be camouflaged. The fact that this nauseating odor evolves between the time of production of the composition and the time of use of the product represents a real challenge for the formulator. This greatly complicates the development of a cosmetic composition acceptable to the sensitive nose of the consumer.

In particular, such a development may require the use of several fragrances or fragrances in larger quantities, which in particular increases the risk of skin irritation.

Thus, there remains a need for highly pure volatile alkane mixtures with a well-defined and adjustable composition obtained from biosourced materials in order to develop their use as substitutes for volatile products from petrochemicals in cosmetic compositions.

Surprisingly, the Applicant has developed a process for producing a volatile mixture of alkanes having the following characteristics.

The process for producing a volatile mixture of alkanes according to the invention also makes it possible to produce volatile mixtures of alkanes having a biodegradability of at least 50% according to the OECD 301F method.

Advantageously, the process for producing a volatile mixture of alkanes according to the invention can be implemented under improved safety conditions, in particular avoiding the operator having to handle low flash point alkanes to produce a mixture of the latter.

Furthermore, the process for preparing the volatile mixture of alkanes according to the invention allows the preparation of cosmetic compositions which can have a naturalness index greater than or equal to 90%, more advantageously greater than or equal to 95%.

The invention relates to a process for preparing a volatile mixture of alkanes comprising the following steps:
Step 1: production of a Guerbet alcohol by Guerbet synthesis from a biosourced linear or branched alcohol,
Step 2: Add to the Guerbet alcohol formed in step 1 at least one biosourced linear or branched alcohol to form a mixture of biosourced alcohols,
Step 3: obtaining a volatile mixture of alkanes consisting of the branched alkane from Guerbet alcohol and at least one alkane from the at least one biosourced alcohol added in step 2 by a dehydration reaction followed by a hydrogenation reaction of the mixture of biosourced alcohols formed in step 2.

In one embodiment, the invention relates to a process for preparing a volatile mixture of alkanes comprising the following steps:
Step 1: the production of a Guerbet alcohol by a Guerbet synthesis from a biosourced linear or branched alcohol with a mass purity greater than or equal to 80%,
Step 2: Add to the Guerbet alcohol formed in step 1 at least one biosourced linear or branched alcohol to form a mixture of biosourced alcohols,
Step 3: obtaining a volatile mixture of alkanes consisting of the branched alkane from Guerbet alcohol and at least one alkane from the at least one biosourced alcohol added in step 2 by a dehydration reaction followed by a hydrogenation reaction of the mixture of biosourced alcohols formed in step 2.

The invention also relates to a process for preparing a volatile mixture of alkanes comprising the following steps:
Step 1: production of a Guerbet alcohol by Guerbet synthesis from a linear or branched biosourced alcohol in C≤10,
Step 2: Add to the Guerbet alcohol formed in step 1 at least one linear or branched biosourced alcohol in C>10 to form a mixture of biosourced alcohols,
Step 3: obtaining a volatile mixture of alkanes consisting of the branched alkane from Guerbet alcohol and at least one alkane from the at least one biosourced alcohol added in step 2 by a dehydration reaction followed by a hydrogenation reaction of the mixture of biosourced alcohols formed in step 2.

The invention also relates to a process for preparing a volatile mixture of alkanes comprising the following steps:
Step 1: production of a Guerbet alcohol by Guerbet synthesis from a linear or branched biosourced C≤10 alcohol with a mass purity greater than or equal to 80%
Step 2: Add to the Guerbet alcohol formed in step 1 at least one linear or branched biosourced alcohol in C>10 to form a mixture of biosourced alcohols
Step 3: obtaining a volatile mixture of alkanes consisting of the branched alkane from Guerbet alcohol and at least one alkane from the at least one biosourced alcohol added in step 2 by a dehydration reaction followed by a hydrogenation reaction of the mixture of biosourced alcohols formed in step 2.

In particular, the invention relates to a process for preparing a volatile mixture of alkanes comprising the following steps:
Step 1: production of a Guerbet alcohol by Guerbet synthesis from a biosourced branched alcohol in C≤10,
Step 2: Add to the Guerbet alcohol formed in step 1 at least one linear or branched biosourced alcohol in C>10 to form a mixture of biosourced alcohols
Step 3: obtaining a volatile mixture of alkanes consisting of the branched alkane derived from Guerbet alcohol and at least one linear or branched alkane derived from the at least one biosourced alcohol added in step 2 by a dehydration reaction followed by a hydrogenation reaction of the mixture of biosourced alcohols formed in step 2.

More particularly, the invention relates to a process for preparing a volatile mixture of alkanes comprising the following steps:
Step 1: production of a Guerbet alcohol by Guerbet synthesis from a biosourced branched alcohol in C≤10 with a mass purity greater than or equal to 80%
Step 2: Add to the Guerbet alcohol formed in step 1 at least one linear or branched biosourced alcohol in C>10 to form a mixture of biosourced alcohols
Step 3: obtaining a volatile mixture of alkanes consisting of the branched alkane derived from Guerbet alcohol and at least one linear or branched alkane derived from the at least one biosourced alcohol added in step 2 by a dehydration reaction followed by a hydrogenation reaction of the mixture of biosourced alcohols formed in step 2.

The invention relates more particularly to a process for preparing a volatile mixture of alkanes comprising the following steps:
Step 1: Production of a C10 Guerbet alcohol, 2-isopropyl-5-methylhexan-1-ol (CAS 2051-33-4), by Guerbet synthesis from isoamyl alcohol (CAS 123-51-3) with a mass purity greater than or equal to 80%
Step 2: Add to the 2-isopropyl-5-methylhexan-1-ol (CAS 2051-33-4) formed in step 1 at least one linear or branched biosourced alcohol in C>10 to form a mixture of biosourced alcohols
Step 3: obtaining a volatile mixture of alkanes consisting of 2,3,6-trimethylheptane (CAS 4032-93-3) and at least one linear or branched C>10 alkane by a dehydration reaction followed by a hydrogenation reaction of the mixture of biosourced alcohols formed in step 2

Advantageously, the volatile mixture of alkanes obtained by the process according to the invention has a biodegradability of at least 50% according to the OECD 301F method.

Step 1 of the process for preparing a volatile mixture of alkanes according to the invention consists of the production of a Guerbet alcohol by a Guerbet synthesis from a biosourced linear or branched alcohol with a mass purity greater than or equal to 80%.

In one embodiment, the process for preparing a volatile mixture of alkanes according to the invention is characterized in that the Guerbet alcohol obtained during step 1 of the process is a compound of formula R—CH₂—CH₂—CH(R)—CH₂—OH resulting from a Guerbet synthesis from a biosourced linear or branched alcohol of formula R—CH₂—CH₂—OH, R being a linear or branched alkyl chain comprising from 1 to 6 carbon atoms.

In one embodiment, the process for preparing a volatile mixture of alkanes according to the invention is characterized in that the Guerbet alcohol obtained during step 1 of the process is a compound of formula R′—CH₂—CH(R″)—CH(R′)—CH(R″)—OH resulting from a Guerbet synthesis from a biosourced branched alcohol of formula R′—CH₂—CH(R″)—OH, R′ and R″ being identical or different, linear or branched alkyl chains, comprising from 1 to 6 carbon atoms.

In one embodiment, the process for preparing a volatile mixture of alkanes according to the invention is characterized in that step 1 of the process is carried out from an alcohol of plant, bacterial or animal origin.

In one embodiment, the process for preparing a volatile mixture of alkanes according to the invention is characterized in that step 1 of the process is carried out from an alcohol of plant origin.

In one embodiment, the process for preparing a volatile mixture of alkanes according to the invention is characterized in that step 1 of the process is carried out from a biosourced linear or branched alcohol in C≤10.

In one embodiment, the process for preparing a volatile mixture of alkanes according to the invention is characterized in that step 1 of the process is carried out from a biosourced linear or branched C≤10 alcohol chosen from the group consisting of isoamyl alcohol (CAS 123-51-3), isobutyl alcohol (CAS 78-83-1), 1-hexanol (CAS 111-27-3) and 1-heptanol (CAS 111-70-6).

In one embodiment, the process for preparing a volatile mixture of alkanes according to the invention is characterized in that step 1 of the process is carried out from isoamyl alcohol (CAS 123-51-3).

In one embodiment, the process for preparing a volatile mixture of alkanes according to the invention is characterized in that step 1 of the process is carried out from 1-hexanol (CAS 111-27-3).

In one embodiment, the process for preparing a volatile mixture of alkanes according to the invention is characterized in that step 1 of the process is carried out using at least one base and at least one catalyst.

In one embodiment, the process for preparing a volatile mixture of alkanes according to the invention is characterized in that step 1 of the process is carried out using at least one base chosen from the group consisting of KOH, NaOH, NaOCH₃ and KOCH₃.

In one embodiment, the process for preparing a volatile mixture of alkanes according to the invention is characterized in that step 1 of the process is carried out using at least one catalyst chosen from the group consisting of copper derivatives such as PriCat Cu 50/8, a group VIII metal such as nickel, palladium, platinum, their derivatives and their mixtures.

In one embodiment, the process for preparing a volatile mixture of alkanes according to the invention is characterized in that step 1 of the process is carried out using at least the PriCat Cu 50/8 catalyst.

In one embodiment, the process for preparing a volatile mixture of alkanes according to the invention is characterized in that step 1 of the process is carried out at temperatures between 150 and 300° C.

In one embodiment, the process for preparing a volatile mixture of alkanes according to the invention is characterized in that step 1 of the process is carried out at temperatures between 170 and 260° C.

In one embodiment, the process for preparing a volatile mixture of alkanes according to the invention is characterized in that step 1 of the process is carried out at temperatures between 180 and 230° C.

In one embodiment, the process for preparing a volatile mixture of alkanes according to the invention is characterized in that step 1 of the process is carried out at pressures between 1 and 15 bars.

In one embodiment, the process for preparing a volatile mixture of alkanes according to the invention is characterized in that step 1 of the process is carried out at atmospheric pressure.

In one embodiment, the process for preparing a volatile mixture of alkanes according to the invention is characterized in that step 1 of the process is carried out for a duration of between 10 and 24 hours.

In one embodiment, the process for preparing a volatile mixture of alkanes according to the invention is characterized in that step 1 of the process is carried out for a duration of 24 hours.

In one embodiment, the process for preparing a volatile mixture of alkanes according to the invention is characterized in that step 1 of the process further comprises a purification step by distillation to remove impurities.

In one embodiment, the process for preparing a volatile mixture of alkanes according to the invention is characterized in that step 1 of the process can be carried out according to the conditions described in patent applications EP2913319A1, WO02/24616A1, WO2011/054483A1, WO2017/093473A1 and WO2020/093127A1 in particular.

In one embodiment, the process for preparing a volatile mixture of alkanes according to the invention is characterized in that step 1 of the process makes it possible to obtain a mass percentage of Guerbet alcohol greater than or equal to 75%.

In one embodiment, the process for preparing a volatile mixture of alkanes according to the invention is characterized in that step 1 of the process makes it possible to obtain a mass percentage of Guerbet alcohol greater than or equal to 90%.

In one embodiment, the process for preparing a volatile mixture of alkanes according to the invention is characterized in that step 1 of the process makes it possible to obtain a branched alcohol whose percentage of carbon 14 (¹⁴C) is greater than or equal to 75% determined according to one of the following standards ASTM D6866, EN 16640 or EN 16785-1.

In one embodiment, the process for preparing a volatile mixture of alkanes according to the invention is characterized in that step 1 of the process makes it possible to obtain a branched alcohol whose percentage of carbon 14 (¹⁴C) is greater than or equal to 90% determined according to one of the following standards ASTM D6866, EN 16640 or EN 16785-1.

In one embodiment, the process for preparing a volatile mixture of alkanes according to the invention is characterized in that step 1 of the process makes it possible to obtain a branched alcohol whose percentage of carbon 14 (¹⁴C) is 100% determined according to one of the following standards ASTM D6866, EN 16640 or EN 16785-1.

In one embodiment, the process for preparing a volatile mixture of alkanes according to the invention is characterized in that the Guerbet alcohol obtained during step 1 of the process is 2-isopropyl-5-methylhexan-1-ol.

In one embodiment, the process for preparing a volatile mixture of alkanes according to the invention is characterized in that the Guerbet alcohol obtained during step 1 of the process is 2-butyloctan-1-ol.

Step 2 of the process for preparing a volatile mixture of alkanes according to the invention consists in adding to the Guerbet alcohol obtained during step 1 at least one biosourced linear or branched alcohol to form a mixture of biosourced alcohols.

In one embodiment, the process for preparing a volatile mixture of alkanes according to the invention is characterized in that the at least one alcohol added to the Guerbet alcohol during step 2 of the process is a biosourced linear alcohol.

In one embodiment, the process for preparing a volatile mixture of alkanes according to the invention is characterized in that the at least one alcohol added to the Guerbet alcohol during step 2 of the process is a biosourced linear alcohol in C>10.

In one embodiment, the process for preparing a volatile mixture of alkanes according to the invention is characterized in that the at least one alcohol added to the Guerbet alcohol during step 2 of the process is a biosourced linear alcohol in C>10 chosen from the group consisting of n-decanol, n-dodecanol and n-tetradecanol.

In one embodiment, the process for preparing a volatile mixture of alkanes according to the invention is characterized in that the at least one alcohol added to the Guerbet alcohol during step 2 of the process is n-decanol.

In one embodiment, the process for preparing a volatile mixture of alkanes according to the invention is characterized in that the at least one alcohol added to the Guerbet alcohol during step 2 of the process is n-dodecanol.

In one embodiment, the process for preparing a volatile mixture of alkanes according to the invention is characterized in that the at least one alcohol added to the Guerbet alcohol during step 2 of the process is n-tetradecanol.

In one embodiment, the process for preparing a volatile mixture of alkanes according to the invention is characterized in that the alcohol used in step 1 and the alcohol added to the Guerbet alcohol in step 2 are of plant origin.

In one embodiment, the process for preparing a volatile mixture of alkanes according to the invention is characterized in that the alcohol used in step 1 is isoamyl alcohol and the at least one alcohol added to the Guerbet alcohol in step 2 is n-dodecanol.

In one embodiment, the process for preparing a volatile mixture of alkanes according to the invention is characterized in that the alcohol used in step 1 is 1-hexanol and the at least one alcohol added to the Guerbet alcohol in step 2 is n-dodecanol.

In one embodiment, the process for preparing a volatile mixture of alkanes according to the invention is characterized in that the at least one alcohol added to the Guerbet alcohol during step 2 of the process is a biosourced branched alcohol.

In one embodiment, the process for preparing a volatile mixture of alkanes according to the invention is characterized in that the at least one alcohol added to the Guerbet alcohol during step 2 of the process is a biosourced branched alcohol resulting from a Guerbet synthesis.

In one embodiment, the process for preparing a volatile mixture of alkanes according to the invention is characterized in that the at least one alcohol added to the

Guerbet alcohol during step 2 of the process is a biosourced branched alcohol resulting from a Guerbet synthesis chosen from the group consisting of 2-butyloctan-1-ol and 2-isopropyl-5-methylhexan-1-ol.

In one embodiment, the process for preparing a volatile mixture of alkanes according to the invention is characterized in that the at least one alcohol resulting from a Guerbet synthesis added to the Guerbet alcohol during step 2 of the process is 2-butyloctan-1-ol.

In one embodiment, the process for preparing a volatile mixture of alkanes according to the invention is characterized in that the at least one alcohol resulting from a Guerbet synthesis added to the Guerbet alcohol during step 2 of the process is 2-isopropyl-5-methylhexan-1-ol.

In one embodiment, the process for preparing a volatile mixture of alkanes according to the invention is characterized in that the at least one alcohol added to the Guerbet alcohol during step 2 of the process is a biosourced branched alcohol in C>10.

In one embodiment, the process for preparing a volatile mixture of alkanes according to the invention is characterized in that two biosourced alcohols are added to the Guerbet alcohol during step 2 of the process.

In one embodiment, the process for preparing a volatile mixture of alkanes according to the invention is characterized in that two biosourced linear alcohols are added to the Guerbet alcohol during step 2 of the process.

In one embodiment, the process for preparing a volatile mixture of alkanes according to the invention is characterized in that two biosourced branched alcohols are added to the Guerbet alcohol during step 2 of the process.

In one embodiment, the process for preparing a volatile mixture of alkanes according to the invention is characterized in that a biosourced linear alcohol and a biosourced branched alcohol are added to the Guerbet alcohol during step 2 of the process.

In one embodiment, the process for preparing a volatile mixture of alkanes according to the invention is characterized in that 1-dodecanol and 2-butyloctan-1-ol are added to the Guerbet alcohol during step 2 of the process.

In one embodiment, the process for preparing a volatile mixture of alkanes according to the invention is characterized in that the mixture of biosourced alcohols formed during step 2 of the process comprises at least 20% by mass of the Guerbet alcohol obtained during step 1 relative to the total mass of the mixture.

In one embodiment, the process for preparing a volatile mixture of alkanes according to the invention is characterized in that the mixture of biosourced alcohols formed during step 2 of the process comprises at least 50% by mass of Guerbet alcohol relative to the total mass of the mixture.

In one embodiment, the process for preparing a volatile mixture of alkanes according to the invention is characterized in that the mass percentage of Guerbet alcohol is between 20% and 95% relative to the total mass of the mixture of biosourced alcohols.

In one embodiment, the process for preparing a volatile mixture of alkanes according to the invention is characterized in that the mass percentage of Guerbet alcohol is between 20% and 70% relative to the total mass of the mixture of biosourced alcohols.

In one embodiment, the process for preparing a volatile mixture of alkanes according to the invention is characterized in that the mass percentage of Guerbet alcohol is between 51% and 95% relative to the total mass of the mixture of biosourced alcohols.

In one embodiment, the process for preparing a volatile mixture of alkanes according to the invention is characterized in that the mass percentage of Guerbet alcohol is between 30% and 60% relative to the total mass of the mixture of biosourced alcohols.

In one embodiment, the process for preparing a volatile mixture of alkanes according to the invention is characterized in that the mass percentage of Guerbet alcohol is between 40% and 70% relative to the total mass of the mixture of biosourced alcohols.

In one embodiment, the process for preparing a volatile mixture of alkanes according to the invention is characterized in that the mass percentage of Guerbet alcohol is between 50% and 80% relative to the total mass of the mixture of biosourced alcohols.

In one embodiment, the process for preparing a volatile mixture of alkanes according to the invention is characterized in that the mass percentage of Guerbet alcohol is between 30% and 50% relative to the total mass of the mixture of biosourced alcohols.

In one embodiment, the process for preparing a volatile mixture of alkanes according to the invention is characterized in that the mass percentage of Guerbet alcohol is between 40% and 60% relative to the total mass of the mixture of biosourced alcohols.

In one embodiment, the process for preparing a volatile mixture of alkanes according to the invention is characterized in that the mass percentage of Guerbet alcohol is between 50% and 70% relative to the total mass of the mixture of biosourced alcohols.

In one embodiment, the process for preparing a volatile mixture of alkanes according to the invention is characterized in that the mass percentage of Guerbet alcohol is between 60% and 80% relative to the total mass of the mixture of biosourced alcohols.

In one embodiment, the process for preparing a volatile mixture of alkanes according to the invention is characterized in that the mass percentage of Guerbet alcohol is between 70% and 90% relative to the total mass of the mixture of biosourced alcohols.

Step 3 of the process for preparing a volatile mixture of alkanes according to the invention consists in obtaining a volatile mixture of alkanes consisting of the branched alkane resulting from the Guerbet alcohol obtained during step 1 and at least one alkane resulting from the at least one biosourced alcohol added in step 2 by a dehydration reaction followed by a hydrogenation reaction of the mixture of biosourced alcohols formed in step 2.

In one embodiment, the process for preparing a volatile mixture of alkanes according to the invention is characterized in that the dehydration reaction of step 3 of the process is carried out using a catalyst.

In one embodiment, the process for preparing a volatile mixture of alkanes according to the invention is characterized in that the dehydration reaction of step 3 of the process is carried out using a catalyst chosen from the group consisting of alumina and its derivatives.

In one embodiment, the process for preparing a volatile mixture of alkanes according to the invention is characterized in that the dehydration reaction of step 3 of the process is carried out using a supported catalyst.

In one embodiment, the process for preparing a volatile mixture of alkanes according to the invention is characterized in that the dehydration reaction of step 3 of the process is carried out using a catalytic support chosen from the group consisting of silica wool and its derivatives.

In one embodiment, the process for preparing a volatile mixture of alkanes according to the invention is characterized in that the dehydration reaction of step 3 of the process is carried out at temperatures between 200 and 380° C.

In one embodiment, the process for preparing a volatile mixture of alkanes according to the invention is characterized in that the dehydration reaction of step 3 of the process is carried out at temperatures between 250 and 350° C.

In one embodiment, the process for preparing a volatile mixture of alkanes according to the invention is characterized in that the dehydration reaction of step 3 of the process is carried out at approximately 330° C.

In one embodiment, the process for preparing a volatile mixture of alkanes according to the invention is characterized in that the dehydration reaction of step 3 of the process is carried out under an inert atmosphere.

In one embodiment, the process for preparing a volatile mixture of alkanes according to the invention is characterized in that the dehydration reaction of step 3 of the process makes it possible to obtain a conversion into alkenes greater than or equal to 95%.

In one embodiment, the process for preparing a volatile mixture of alkanes according to the invention is characterized in that the dehydration reaction of step 3 of the process makes it possible to obtain a conversion into alkenes greater than or equal to 99%.

In one embodiment, the process for preparing a volatile mixture of alkanes according to the invention is characterized in that the dehydration reaction of step 3 of the process is carried out continuously.

In one embodiment, the process for preparing a volatile mixture of alkanes according to the invention is characterized in that the dehydration reaction of step 3 of the process does not comprise a purification step.

In one embodiment, the process for preparing a volatile mixture of alkanes according to the invention is characterized in that the hydrogenation reaction of step 3 of the process is carried out using a catalyst.

In one embodiment, the process for preparing a volatile mixture of alkanes according to the invention is characterized in that the hydrogenation reaction of step 3 of the process is carried out using a catalyst comprising at least one metal from group VIII such as palladium, nickel, platinum, their derivatives and their mixtures.

In one embodiment, the process for preparing a volatile mixture of alkanes according to the invention is characterized in that the hydrogenation reaction of step 3 of the process is carried out using a supported catalyst.

In one embodiment, the process for preparing a volatile mixture of alkanes according to the invention is characterized in that the hydrogenation reaction of step 3 of the process is carried out using a catalytic support chosen from the group consisting of silica wool and its derivatives.

In one embodiment, the process for preparing a volatile mixture of alkanes according to the invention is characterized in that the hydrogenation reaction of step 3 of the process is carried out at temperatures between 100 and 250° C.

In one embodiment, the process for preparing a volatile mixture of alkanes according to the invention is characterized in that the hydrogenation reaction of step 3 of the process is carried out at temperatures between 150 and 250° C.

In one embodiment, the process for preparing a volatile mixture of alkanes according to the invention is characterized in that the hydrogenation reaction of step 3 of the process is carried out at temperatures between 170 and 220° C.

In one embodiment, the process for preparing a volatile mixture of alkanes according to the invention is characterized in that the hydrogenation reaction of step 3 of the process is carried out by heating to at least one temperature level.

In one embodiment, the process for preparing a volatile mixture of alkanes according to the invention is characterized in that the hydrogenation reaction of step 3 of the process is carried out by heating at two different temperature levels.

In one embodiment, the process for preparing a volatile mixture of alkanes according to the invention is characterized in that the hydrogenation reaction of step 3 of the process is carried out by heating to a temperature level of approximately 180° C. and a temperature level of approximately 200° C.

In one embodiment, the process for preparing a volatile mixture of alkanes according to the invention is characterized in that the hydrogenation reaction of step 3 of the process is carried out under an inert atmosphere.

In one embodiment, the process for preparing a volatile mixture of alkanes according to the invention is characterized in that the hydrogenation reaction of step 3 of the process is carried out under an inert nitrogen atmosphere.

In one embodiment, the process for preparing a volatile mixture of alkanes according to the invention is characterized in that the hydrogenation reaction of step 3 of the process is preceded by at least one nitrogen inerting cycle in the reactor.

In one embodiment, the process for preparing a volatile mixture of alkanes according to the invention is characterized in that the hydrogenation reaction of step 3 of the process is preceded by inerting with 15 bars of nitrogen in the reactor.

In one embodiment, the process for preparing a volatile mixture of alkanes according to the invention is characterized in that the hydrogenation reaction of step 3 of the process is preceded by several nitrogen inerting cycles in the reactor.

In one embodiment, the process for preparing a volatile mixture of alkanes according to the invention is characterized in that the hydrogenation reaction of step 3 of the process is preceded by three nitrogen inerting cycles in the reactor.

In one embodiment, the process for preparing a volatile mixture of alkanes according to the invention is characterized in that the hydrogenation reaction of step 3 of the process is preceded by three inerting cycles with 5 bars of nitrogen in the reactor.

In one embodiment, the process for preparing a volatile mixture of alkanes according to the invention is characterized in that the hydrogenation reaction of step 3 of the process is carried out by at least one addition of hydrogen to the reactor.

In one embodiment, the process for preparing a volatile mixture of alkanes according to the invention is characterized in that the hydrogenation reaction of step 3 of the process is carried out by adding 2 to 15 bars of hydrogen to the reactor.

In one embodiment, the process for preparing a volatile mixture of alkanes according to the invention is characterized in that the hydrogenation reaction of step 3 of the process is carried out by adding at least 5 bars of hydrogen to the reactor.

In one embodiment, the process for preparing a volatile mixture of alkanes according to the invention is characterized in that the hydrogenation reaction of step 3 of the process is carried out by adding at least 10 bars of hydrogen to the reactor.

In one embodiment, the process for preparing a volatile mixture of alkanes according to the invention is characterized in that the hydrogenation reaction of step 3 of the process is carried out for a period of 3 to 12 hours.

In one embodiment, the process for preparing a volatile mixture of alkanes according to the invention is characterized in that the hydrogenation reaction of step 3 of the process is carried out for a period of 4 to 8 hours.

In one embodiment, the process for preparing a volatile mixture of alkanes according to the invention is characterized in that the hydrogenation reaction of step 3 of the process is carried out for a period of 5 hours.

In one embodiment, the process for preparing a volatile mixture of alkanes according to the invention is characterized in that the hydrogenation reaction of step 3 of the process makes it possible to obtain a conversion into a volatile mixture of alkanes greater than or equal to 95%.

In one embodiment, the process for preparing a volatile mixture of alkanes according to the invention is characterized in that the hydrogenation reaction of step 3 of the process makes it possible to obtain a conversion into a volatile mixture of alkanes greater than or equal to 99%.

In one embodiment, the process for preparing a volatile mixture of alkanes according to the invention is characterized in that the volatile mixture of alkanes obtained in step 3 contains at least 20% by mass of the branched alkane derived from Guerbet alcohol and at least 20% by mass of at least one alkane derived from the at least one biosourced alcohol added in step 2, relative to the total mass of the volatile mixture of alkanes.

In one embodiment, the process for preparing a volatile mixture of alkanes according to the invention is characterized in that the volatile mixture of alkanes obtained in step 3 comprises at least 20% by mass of the branched alkane derived from Guerbet alcohol relative to the total mass of the mixture.

In one embodiment, the process for preparing a volatile mixture of alkanes according to the invention is characterized in that the volatile mixture of alkanes obtained in step 3 comprises at least 50% by mass of the branched alkane derived from Guerbet alcohol relative to the total mass of the mixture.

In one embodiment, the process for preparing a volatile mixture of alkanes according to the invention is characterized in that the mass percentage of the branched alkane derived from Guerbet alcohol is between 20% and 95% relative to the total mass of the volatile mixture of alkanes.

In one embodiment, the process for preparing a volatile mixture of alkanes according to the invention is characterized in that the mass percentage of the branched alkane derived from Guerbet alcohol is between 20% and 70% relative to the total mass of the volatile mixture of alkanes.

In one embodiment, the process for preparing a volatile mixture of alkanes according to the invention is characterized in that the mass percentage of the branched alkane derived from Guerbet alcohol is between 51% and 95% relative to the total mass of the volatile mixture of alkanes.

In one embodiment, the process for preparing a volatile mixture of alkanes according to the invention is characterized in that the mass percentage of the branched alkane derived from Guerbet alcohol is between 30% and 60% relative to the total mass of the volatile mixture of alkanes.

In one embodiment, the process for preparing a volatile mixture of alkanes according to the invention is characterized in that the mass percentage of the branched alkane derived from Guerbet alcohol is between 40% and 70% relative to the total mass of the volatile mixture of alkanes.

In one embodiment, the process for preparing a volatile mixture of alkanes according to the invention is characterized in that the mass percentage of the branched alkane derived from Guerbet alcohol is between 50% and 80% relative to the total mass of the volatile mixture of alkanes.

In one embodiment, the process for preparing a volatile mixture of alkanes according to the invention is characterized in that the mass percentage of the branched alkane derived from Guerbet alcohol is between 30% and 50% relative to the total mass of the volatile mixture of alkanes.

In one embodiment, the process for preparing a volatile mixture of alkanes according to the invention is characterized in that the mass percentage of the branched alkane derived from Guerbet alcohol is between 40% and 60% relative to the total mass of the volatile mixture of alkanes.

In one embodiment, the process for preparing a volatile mixture of alkanes according to the invention is characterized in that the mass percentage of the branched alkane derived from Guerbet alcohol is between 50% and 70% relative to the total mass of the volatile mixture of alkanes.

In one embodiment, the process for preparing a volatile mixture of alkanes according to the invention is characterized in that the mass percentage of the branched alkane derived from Guerbet alcohol is between 60% and 80% relative to the total mass of the volatile mixture of alkanes.

In one embodiment, the process for preparing a volatile mixture of alkanes according to the invention is characterized in that the mass percentage of the branched alkane derived from Guerbet alcohol is between 70% and 90% relative to the total mass of the volatile mixture of alkanes.

In one embodiment, the process for preparing a volatile mixture of alkanes according to the invention is characterized in that the volatile mixture of alkanes obtained in step 3 contains at least 5-methylundecane.

In one embodiment, the process for preparing a volatile mixture of alkanes according to the invention is characterized in that the volatile mixture of alkanes obtained in step 3 contains 5-methylundecane and at least one alkane chosen from the group consisting of n-decane, n-dodecane and n-tetradecane.

In one embodiment, the process for preparing a volatile mixture of alkanes according to the invention is characterized in that the volatile mixture of alkanes obtained in step 3 consists of:
50% 5-methylundecane (CAS 1632-70-8)
50% n-dodecane (CAS 112-40-3).

In one embodiment, the process for preparing a volatile mixture of alkanes according to the invention is characterized in that the volatile mixture of alkanes obtained in step 3 contains at least 2,3,6-trimethylheptane.

In one embodiment, the process for preparing a volatile mixture of alkanes according to the invention is characterized in that the volatile mixture of alkanes obtained in step 3 contains 2,3,6-trimethylheptane and at least one alkane chosen from the group consisting of n-decane, n-dodecane and n-tetradecane.

In one embodiment, the process for preparing a volatile mixture of alkanes according to the invention is characterized in that the volatile mixture of alkanes obtained in step 3 consists of 2,3,6-trimethylheptane and n-decane.

In one embodiment, the process for preparing a volatile mixture of alkanes according to the invention is characterized in that the volatile mixture of alkanes obtained in step 3 consists of 2,3,6-trimethylheptane and n-dodecane.

In one embodiment, the process for preparing a volatile mixture of alkanes according to the invention is characterized in that the volatile mixture of alkanes obtained in step 3 contains at least 20% by mass of 2,3,6-trimethylheptane and at least 20% by mass of at least one linear alkane chosen from the group consisting of n-decane, n-dodecane and n-tetradecane, relative to the total mass of the volatile mixture of alkanes.

In one embodiment, the process for preparing a volatile mixture of alkanes according to the invention is characterized in that the volatile mixture of alkanes obtained in step 3 contains at least 20% by mass of 2,3,6-trimethylheptane and at least 20% by mass of n-dodecane, relative to the total mass of the volatile mixture of alkanes.

In one embodiment, the process for preparing a volatile mixture of alkanes according to the invention is characterized in that the volatile mixture of alkanes obtained in step 3 consists of:
20 to 80% of 2,3,6-trimethylheptane (CAS 4032-93-3)
20 to 80% n-dodecane (CAS 112-40-3).

In one embodiment, the process for preparing a volatile mixture of alkanes according to the invention is characterized in that the volatile mixture of alkanes obtained in step 3 consists of:
20 to 70% of 2,3,6-trimethylheptane (CAS 4032-93-3)
30 to 80% n-dodecane (CAS 112-40-3).

In one embodiment, the process for preparing a volatile mixture of alkanes according to the invention is characterized in that the volatile mixture of alkanes obtained in step 3 consists of:
30% 2,3,6-trimethylheptane (CAS 4032-93-3)
70% n-dodecane (CAS 112-40-3).

In one embodiment, the process for preparing a volatile mixture of alkanes according to the invention is characterized in that the volatile mixture of alkanes obtained in step 3 consists of:
35% 2,3,6-trimethylheptane (CAS 4032-93-3)
65% n-dodecane (CAS 112-40-3).

In one embodiment, the process for preparing a volatile mixture of alkanes according to the invention is characterized in that the volatile mixture of alkanes obtained in step 3 consists of:
40% 2,3,6-trimethylheptane (CAS 4032-93-3)
60% n-dodecane (CAS 112-40-3).

In one embodiment, the process for preparing a volatile mixture of alkanes according to the invention is characterized in that the volatile mixture of alkanes obtained in step 3 consists of:
45% 2,3,6-trimethylheptane (CAS 4032-93-3)
55% n-dodecane (CAS 112-40-3).

In one embodiment, the process for preparing a volatile mixture of alkanes according to the invention is characterized in that the volatile mixture of alkanes obtained in step 3 consists of:
60% 2,3,6-trimethylheptane (CAS 4032-93-3)
40% n-dodecane (CAS 112-40-3).

In one embodiment, the process for preparing a volatile mixture of alkanes according to the invention is characterized in that the volatile mixture of alkanes obtained in step 3 consists of:
70% 2,3,6-trimethylheptane (CAS 4032-93-3)
30% n-dodecane (CAS 112-40-3).

In one embodiment, the process for preparing a volatile mixture of alkanes according to the invention is characterized in that the volatile mixture of alkanes obtained in step 3 consists of:
90% 2,3,6-trimethylheptane (CAS 4032-93-3)
10% n-dodecane (CAS 112-40-3).

In one embodiment, the process for preparing a volatile mixture of alkanes according to the invention is characterized in that the volatile mixture of alkanes obtained in step 3 consists of 2,3,6-trimethylheptane and n-tetradecane.

In one embodiment, the process for preparing a volatile mixture of alkanes according to the invention is characterized in that the volatile mixture of alkanes obtained in step 3 consists of a branched alkane derived from the Guerbet alcohol obtained in step 1 and at least one branched alkane derived from the at least one biosourced alcohol added in step 2.

In one embodiment, the process for preparing a volatile mixture of alkanes according to the invention is characterized in that the volatile mixture of alkanes obtained in step 3 consists of 2,3,6-trimethylheptane and 5-methylundecane.

In one embodiment, he process for preparing a volatile mixture of alkanes according to the invention is characterized in that the volatile mixture of alkanes obtained in step 3 consists of a branched alkane derived from the Guerbet alcohol obtained in step 1 and at least one branched alkane and one linear alkane derived from at least two biosourced alcohols added in step 2.

In one embodiment, the process for preparing a volatile mixture of alkanes according to the invention is characterized in that the volatile mixture of alkanes obtained in step 3 consists of 2,3,6-trimethylheptane, 5-methylundecane and n-dodecane.

In one embodiment, the process for preparing a volatile mixture of alkanes according to the invention is characterized in that the volatile mixture of alkanes obtained in step 3 consists of:
30% 2,3,6-trimethylheptane (CAS 4032-93-3)
30% 5-methylundecane (CAS 1632-70-8)
40% n-dodecane (CAS 112-40-3).

In one embodiment, the process for preparing a volatile mixture of alkanes according to the invention is characterized in that the flash point of the volatile mixture of alkanes obtained in step 3 is less than or equal to 50° C.

In one embodiment, the process for preparing a volatile mixture of alkanes according to the invention is characterized in that the flash point of the volatile mixture of alkanes obtained in step 3 is between 45° C. and 50° C.

In one embodiment, the process for preparing a volatile mixture of alkanes according to the invention is characterized in that the flash point of the volatile mixture of alkanes obtained in step 3 is less than or equal to 45° C.

In one embodiment, the process for preparing a volatile mixture of alkanes according to the invention is characterized in that the flash point of the volatile mixture of alkanes obtained in step 3 is between 40° C. and 45° C.

In one embodiment, the process for preparing a volatile mixture of alkanes according to the invention is characterized in that the flash point of the volatile mixture of alkanes obtained in step 3 is less than or equal to 40° C.

In one embodiment, the process for preparing a volatile mixture of alkanes according to the invention is characterized in that the flash point of the volatile mixture of alkanes obtained in step 3 is between 35° C. and 40° C.

In one embodiment, the process for preparing a volatile mixture of alkanes according to the invention is characterized in that the volatile mixture of alkanes obtained in step 3 has a percentage of carbon 14 (¹⁴C) greater than or equal to 75% determined according to one of the following standards ASTM D6866, EN 16640 or EN 16785-1.

In one embodiment, the process for preparing a volatile mixture of alkanes according to the invention is characterized in that the volatile mixture of alkanes obtained in step 3 has a percentage of carbon 14 (¹⁴C) greater than or equal to 90% determined according to one of the following standards ASTM D6866, EN 16640 or EN 16785-1.

In one embodiment, the process for preparing a volatile mixture of alkanes according to the invention is characterized in that the volatile mixture of alkanes obtained in step 3 has a percentage of carbon 14 (¹⁴C) of 100% determined according to one of the following standards ASTM D6866, EN 16640 or EN 16785-1.

In one embodiment, the process for preparing a volatile mixture of alkanes according to the invention is characterized in that the volatile mixture of alkanes obtained in step 3 has a biodegradability of at least 50% according to the OECD 301F method.

In one embodiment, the process for preparing a volatile mixture of alkanes according to the invention is characterized in that the volatile mixture of alkanes obtained in step 3 has a biodegradability of at least 60% according to the OECD 301F method.

In one embodiment, the process for preparing a volatile mixture of alkanes according to the invention is characterized in that the volatile mixture of alkanes obtained in step 3 has a biodegradability of between 50 and 80% according to the OECD 301F method.

In one embodiment, the process for preparing a volatile mixture of alkanes according to the invention is characterized in that the volatile mixture of alkanes obtained in step 3 has a biodegradability of between 50 and 70% according to the OECD 301F method.

In one embodiment, the process for preparing a volatile mixture of alkanes according to the invention is characterized in that the volatile mixture of alkanes obtained in step 3 has a biodegradability of between 50 and 60% according to the OECD 301F method.

In one embodiment, the process for preparing a volatile mixture of alkanes according to the invention is characterized in that the volatile mixture of alkanes obtained in step 3 has a biodegradability of between 60 and 70% according to the OECD 301F method.

In one embodiment, the process for preparing a volatile mixture of alkanes according to the invention is characterized in that the volatile mixture of alkanes obtained in step 3 has a biodegradability of between 70 and 80% according to the OECD 301F method.

Surprisingly, the process for preparing a volatile mixture of alkanes according to the invention is characterized in that the volatile mixture of alkanes obtained in step 3 is deodorized or odorless.

The invention also relates to a process for preparing a cosmetic composition further comprising a step of mixing a volatile mixture of alkanes obtained by the process according to the invention with at least one compound chosen from non-volatile oils, glossy oils, pasty fatty substances, gelling agents, film-forming polymers, waxes, antioxidants, pigments, dyes, surfactants, an aqueous phase, and mixtures thereof.

In one embodiment, the process for preparing a cosmetic composition according to the invention is characterized in that it further comprises a step of mixing a volatile mixture of alkanes obtained by the process according to the invention with at least one non-volatile oil.

For the purposes of the present invention, the term “non-volatile oil” means an oil having a flash point greater than 110° C. measured according to the ATSM D93 standard.

These oils can be of vegetable, mineral or synthetic origin.

These non-volatile oils can be hydrocarbon, silicone, fluorinated or fluorosilicone oils.

For the purposes of the present invention, the term “hydrocarbon oil” means an oil essentially consisting of carbon atoms, hydrogen atoms and optionally oxygen and/or nitrogen atoms.

Among these hydrocarbon oils, mention may be made of linear or branched alkanes of mineral or synthetic origin such as linear or branched alkanes of 17 to 40 carbon atoms, (poly)esters and (poly)ethers, and in particular (poly)esters of C₂-C₂₄ acids (preferably C₆-C₂₀), triglycerides of C₆-C₂₀ fatty acids, vegetable oils, dialkyl carbonates, branched and/or unsaturated fatty acids, branched and/or unsaturated fatty alcohols.

Concerning non-volatile silicone oils, they are notably chosen from the group comprising phenylated silicone oils, non-volatile polydimethylsiloxanes, or derivatives of non-volatile polydimethylsiloxanes and their mixtures.

In a particularly preferred embodiment, the process for preparing a cosmetic composition according to the invention is characterized in that the non-volatile oil is chosen from the group consisting of non-volatile linear or branched alkanes.

Preferably, the non-volatile oil is chosen from the group consisting of linear or branched alkanes of 17 to 40 carbon atoms.

Preferably, non-volatile oils are of plant origin.

In a preferred embodiment, the process for preparing a cosmetic composition according to the invention is characterized in that the non-volatile oil is a vegetable hydrocarbon oil.

The vegetable hydrocarbon oils are notably chosen from the group comprising wheat germ, sunflower, grape seed, sesame, coconut, apricot, castor, corn, avocado, soybean, shea, olive, sweet almond oil, cotton, palm, macadamia, rapeseed, jojoba, hazelnut, poppy, alfalfa, pumpkin, blackcurrant, squash, evening primrose, barley oils, and mixtures thereof.

In one embodiment, the process for preparing a cosmetic composition according to the invention is characterized in that the mass percentage of the non-volatile oil is at least 5% relative to the total mass of the cosmetic composition.

In one embodiment, the process for preparing a cosmetic composition according to the invention is characterized in that the mass percentage of the non-volatile oil is at least 10% relative to the total mass of the cosmetic composition.

In one embodiment, the process for preparing a cosmetic composition according to the invention is characterized in that the mass percentage of the non-volatile oil is between 5% and 25% relative to the total mass of the cosmetic composition.

In one embodiment, the process for preparing a cosmetic composition according to the invention is characterized in that the mass percentage of the non-volatile oil is between 5% and 15% relative to the total mass of the cosmetic composition.

In one embodiment, the process for preparing a cosmetic composition according to the invention is characterized in that the mass percentage of the non-volatile oil is between 10% and 25% relative to the total mass of the cosmetic composition.

In one embodiment, the process for preparing a cosmetic composition according to the invention is characterized in that it further comprises a step of mixing a volatile mixture of alkanes obtained by the process according to the invention with at least one shiny oil.

For the purposes of the present invention, the term “shiny oil” means an oil having intrinsic shine properties. “Shiny oils” are frequently used in cosmetics to ensure a “shiny effect” when applying cosmetic compositions to keratin materials, for example.

In one embodiment, the process for preparing a cosmetic composition according to the invention is characterized in that the shiny oil is a non-volatile oil.

In one embodiment, the method for preparing a cosmetic composition according to the invention is characterized in that the shiny oil is chosen from the group comprising:
polymers such as: polybutylenes, in particular POLYBUTENE® marketed or manufactured by the company ATAMAN, hydrogenated polyisobutylenes, polydecenes and hydrogenated polydecenes, polyfarnesenes, vinylpyrrolidone copolymers, in particular the vinylpyrrolidone/eicosene copolymer, ANTARON V 220® marketed or manufactured by the company ISP.
esters such as esters of linear fatty acids having a total number of carbons greater than 30, aromatic esters, esters of fatty alcohols or branched fatty acids having from 20 to 30 carbon atoms.
ester copolymers such as: dimer dilinoleyl dimer dilinoleate (LUSPLAN DD-DA5® and LUSPLAN DD-DA7®), dilinoleic acid/butanediol copolymer (VISCOPLAST 14436H®), dilinoleic acid/propanediol copolymer and their mixtures.

Preferably, the process for preparing a cosmetic composition according to the invention is characterized in that the shiny oil is of plant origin.

In one embodiment, the process for preparing a cosmetic composition according to the invention is characterized in that the mass percentage of the shiny oil is at least 2% relative to the total mass of the cosmetic composition.

In one embodiment, the process for preparing a cosmetic composition according to the invention is characterized in that the mass percentage of the shiny oil is at least 5% relative to the total mass of the cosmetic composition.

In one embodiment, the process for preparing a cosmetic composition according to the invention is characterized in that the mass percentage of the shiny oil is between 2% and 20% relative to the total mass of the cosmetic composition.

In one embodiment, the process for preparing a cosmetic composition according to the invention is characterized in that the mass percentage of the shiny oil is between 5% and 15% relative to the total mass of the cosmetic composition.

In one embodiment, the process for preparing a cosmetic composition according to the invention is characterized in that the mass percentage of the shiny oil is between 10% and 20% relative to the total mass of the cosmetic composition.

In one embodiment, the method for preparing a cosmetic composition according to the invention is characterized in that the mass percentage of the shiny oil is between 2% and 10% relative to the total mass of the cosmetic composition.

In one embodiment, the process for preparing a cosmetic composition according to the invention is characterized in that the mass percentage of the shiny oil is between 5% and 10% relative to the total mass of the cosmetic composition.

In one embodiment, the process for preparing a cosmetic composition according to the invention is characterized in that it further comprises a step of mixing a volatile mixture of alkanes obtained by the process according to the invention with at least one pasty fatty substance.

For the purposes of the present invention, the term “pasty fatty substance” means a lipophilic fatty compound with a reversible solid/liquid state change, having at room temperature (25° C.) a liquid fraction and a solid fraction.

In other words, the melting temperature of the pasty fat is less than or equal to 25° C.

In one embodiment, the process for preparing a cosmetic composition according to the invention is characterized in that the pasty fatty substance is chosen from the group comprising synthetic pasty fatty substances, animal pasty fatty substances, vegetable pasty fatty substances and their mixtures.

In one embodiment, the process for preparing a cosmetic composition according to the invention is characterized in that the pasty fatty substance is a synthetic pasty fatty substance chosen from the group comprising: polyol ethers, petroleum jelly, vinyl polymers, pentaerythritol esters, polyesters and their mixtures.

In one embodiment, the process for preparing a cosmetic composition according to the invention is characterized in that the animal pasty fatty substance is lanolin.

Preferably, the process for preparing a cosmetic composition according to the invention is characterized in that the pasty fatty substance is a vegetable pasty fatty substance.

In one embodiment, the method for preparing a cosmetic composition according to the invention is characterized in that the vegetable pasty fatty substance is chosen from the group comprising: vegetable butters (avocado butter, shea butter), fatty acid triglycerides and their derivatives, and their mixtures.

In one embodiment, the process for preparing a cosmetic composition according to the invention is characterized in that the mass percentage of the pasty fatty substance is between 0% and 20% relative to the total mass of the cosmetic composition.

In one embodiment, the process for preparing a cosmetic composition according to the invention is characterized in that the mass percentage of the pasty fatty substance is between 0% and 15% relative to the total mass of the cosmetic composition.

In one embodiment, the process for preparing a cosmetic composition according to the invention is characterized in that the mass percentage of the pasty fatty substance is between 0% and 10% relative to the total mass of the cosmetic composition.

In one embodiment, the process for preparing a cosmetic composition according to the invention is characterized in that the mass percentage of the pasty fatty substance is between 0% and 5% relative to the total mass of the cosmetic composition.

In one embodiment, the process for preparing a cosmetic composition according to the invention is characterized in that it further comprises a step of mixing a volatile mixture of alkanes obtained by the process according to the invention with at least one gelling agent.

For the purposes of the present invention, the term gelling agent means compounds which constitute excellent texturizing agents for balancing formulations in terms of smoothness and stickiness.

Furthermore, certain glossy oils such as VISCOPLAST 14436 HR, and compounds marketed under the names LUSPLAN DD-DA5® and LUSPLAN DD-DA7® can also be used as gelling agents.

In one embodiment, the method for preparing a cosmetic composition according to the invention is characterized in that the gelling agent is chosen from the group comprising: organic gelling agents, mineral gelling agents, polymeric gelling agents, and mixtures thereof.

Preferably, the process for preparing a cosmetic composition according to the invention is characterized in that the gelling agent is of plant origin.

In one embodiment, the method for preparing a cosmetic composition according to the invention is characterized in that the gelling agent is an organic gelling agent chosen from the group comprising: ethylcellulose and its derivatives, resins derived from rosin, dextrin esters, fatty acid esters, and mixtures thereof.

In one embodiment, the method for preparing a cosmetic composition according to the invention is characterized in that the gelling agent is a mineral gelling agent chosen from the group comprising: hectorite and its derivatives, pyrogenic silica and its derivatives, and their mixtures.

In one embodiment, the method for preparing a cosmetic composition according to the invention is characterized in that the gelling agent is a polymeric gelling agent chosen from the group comprising: organopolysiloxanes and their derivatives, dimethicone copolymers and their derivatives, polystyrene/polyisoprene copolymers, polystyrene/polybutadiene copolymers, polystyrene/copoly (ethylene-propylene) copolymers, polystyrene/copoly (ethylene-butylene) copolymers, mixtures of copolymers in isododecane such as ethylene/propylene copolymer or butylene/ethylene/styrene copolymer, polyesters and their derivatives, polyamide resins, and mixtures thereof.

In one embodiment, the process for preparing a cosmetic composition according to the invention is characterized in that the gelling agent is dextrin palmitate or one of its derivatives.

In one embodiment, the process for preparing a cosmetic composition according to the invention is characterized in that the gelling agent is a modified hectorite of the BENTONE GEL ISD V® type or one of its equivalents.

In one embodiment, the process for preparing a cosmetic composition according to the invention is characterized in that the gelling agent is a modified hectorite of the VEGELIGHT BENTONE SILK SB® type or one of its equivalents.

In one embodiment, the process for preparing a cosmetic composition according to the invention is characterized in that the gelling agent is a modified hectorite of disteardimonium hectorite type associated with triethyl citrate prepared in the volatile mixture of alkanes obtained in step 3 of the process according to the invention.

In one embodiment, the process for preparing a cosmetic composition according to the invention is characterized in that the gelling agent is a mixture of copolymer in isododecane such as ethylene/propylene copolymer.

In one embodiment, the process for preparing a cosmetic composition according to the invention is characterized in that the gelling agent is the combination of a dimethicone polymer and a mixture of alkanes marketed under the name VEGELIGHT SILK SI 118® by BIOSYNTHIS, or one of its equivalents.

In one embodiment, the process for preparing a cosmetic composition according to the invention is characterized in that the mass percentage of the gelling agent is at least 5% relative to the total mass of the cosmetic composition.

In one embodiment, the process for preparing a cosmetic composition according to the invention is characterized in that the mass percentage of the gelling agent is at least 15% relative to the total mass of the cosmetic composition.

In one embodiment, the process for preparing a cosmetic composition according to the invention is characterized in that the mass percentage of the gelling agent is between 5% and 30% relative to the total mass of the cosmetic composition.

In one embodiment, the process for preparing a cosmetic composition according to the invention is characterized in that the mass percentage of the gelling agent is between 5% and 20% relative to the total mass of the cosmetic composition.

In one embodiment, the process for preparing a cosmetic composition according to the invention is characterized in that the mass percentage of the gelling agent is between 15% and 30% relative to the total mass of the cosmetic composition.

In one embodiment, the process for preparing a cosmetic composition according to the invention is characterized in that the mass percentage of the gelling agent is between 5% and 15% relative to the total mass of the cosmetic composition.

In one embodiment, the process for preparing a cosmetic composition according to the invention is characterized in that the mass percentage of the gelling agent is between 15% and 25% relative to the total mass of the cosmetic composition.

In one embodiment, the process for preparing a cosmetic composition according to the invention is characterized in that the mass percentage of the gelling agent is between 20% and 30% relative to the total mass of the cosmetic composition.

In one embodiment, the process for preparing a cosmetic composition according to the invention is characterized in that it further comprises a step of mixing a volatile mixture of alkanes obtained by the process according to the invention with at least one film-forming polymer.

For the purposes of the present invention, the term film-forming polymer means a polymer capable of forming a macroscopically continuous film, in particular on the lips within the framework of our invention.

In one embodiment, the process for preparing a cosmetic composition according to the invention is characterized in that the film-forming polymer is chosen from the group comprising: acrylic polymers, polyurethanes, polyesters, polyamides, silicone polymers.

In one embodiment, the process for preparing a cosmetic composition according to the invention is characterized in that the film-forming polymer is trimethylsilloxysilicate marketed under the name TMS 803.

In one embodiment, the process for preparing a cosmetic composition according to the invention is characterized in that the mass percentage of the film-forming polymer is at least 1% relative to the total mass of the cosmetic composition.

In one embodiment, the process for preparing a cosmetic composition according to the invention is characterized in that the mass percentage of the film-forming polymer is at least 10% relative to the total mass of the cosmetic composition.

In one embodiment, the process for preparing a cosmetic composition according to the invention is characterized in that the mass percentage of the film-forming polymer is between 1% and 25% relative to the total mass of the cosmetic composition.

In one embodiment, the process for preparing a cosmetic composition according to the invention is characterized in that the mass percentage of the film-forming polymer is between 10% and 25% relative to the total mass of the cosmetic composition.

In one embodiment, the process for preparing a cosmetic composition according to the invention is characterized in that the mass percentage of the film-forming polymer is between 1% and 10% relative to the total mass of the cosmetic composition.

In one embodiment, the process for preparing a cosmetic composition according to the invention is characterized in that the mass percentage of the film-forming polymer is between 10% and 20% relative to the total mass of the cosmetic composition.

In one embodiment, the process for preparing a cosmetic composition according to the invention is characterized in that it further comprises a step of mixing a volatile mixture of alkanes obtained by the process according to the invention with at least one wax.

Waxes play an important role in the design of cosmetic products, particularly lipsticks, because they influence certain essential criteria such as the texture, appearance and/or solidity of the product.

In the context of the present invention, the term “wax” means a lipophilic compound having a reversible solid/liquid state change. In other words, the compound is solid at room temperature (at 25° C.) and has a melting point greater than or equal to 30° C.

Preferably, for the purposes of the present invention, the waxes used have a melting point greater than or equal to 50° C.

In one embodiment, the method for preparing a cosmetic composition according to the invention is characterized in that the at least one wax is chosen from the group consisting of synthetic waxes, mineral waxes, vegetable waxes and animal waxes.

In one embodiment, the method for preparing a cosmetic composition according to the invention is characterized in that the wax is a mineral wax or a synthetic wax chosen from the group comprising: microcrystalline waxes, paraffins, ozokerite, polyethylene waxes, silicone waxes, fluorinated waxes.

By “vegetable wax” is meant a composition comprising at least one partially or completely hydrogenated oil. These vegetable waxes may comprise esters of fatty acids and fatty alcohols, and may further comprise fatty acids, free fatty alcohols, long chain linear alkanes and/or unsaponifiables.

Preferably, within the meaning of the present invention, the waxes used are vegetable waxes.

In one embodiment, the method for preparing a cosmetic composition according to the invention is characterized in that the wax is a vegetable or animal wax chosen from the group comprising: lanolin wax, rice bran wax, carnauba wax, candelilla wax, berry wax, beeswax, sunflower wax, mimosa wax, jojoba wax, castor wax, olive wax.

In one embodiment, the process for preparing a cosmetic composition according to the invention is characterized in that the mass percentage of the wax is at least 2% relative to the total mass of the cosmetic composition.

In one embodiment, the process for preparing a cosmetic composition according to the invention is characterized in that the mass percentage of the wax is at least 5% relative to the total mass of the cosmetic composition.

In one embodiment, the process for preparing a cosmetic composition according to the invention is characterized in that the mass percentage of the wax is between 2% and 20% relative to the total mass of the cosmetic composition.

In one embodiment, the process for preparing a cosmetic composition according to the invention is characterized in that the mass percentage of the wax is between 5% and 15% relative to the total mass of the cosmetic composition.

In one embodiment, the process for preparing a cosmetic composition according to the invention is characterized in that the mass percentage of the wax is between 2% and 10% relative to the total mass of the cosmetic composition.

In one embodiment, the process for preparing a cosmetic composition according to the invention is characterized in that the mass percentage of the wax is between 5% and 10% relative to the total mass of the cosmetic composition.

In one embodiment, the process for preparing a cosmetic composition according to the invention is characterized in that it further comprises a step of mixing a volatile mixture of alkanes obtained by the process according to the invention with at least one antioxidant.

In one embodiment, the method for preparing a cosmetic composition according to the invention is characterized in that the antioxidant is chosen from the group comprising: phenolic antioxidants of the BHA, BHT, DBPC, gallate type, so-called natural antioxidants such as dl-alpha-tocopherol or its derivatives, plant extracts, dibasic lipopeptides such as lysine or arginine, and mixtures thereof.

In one embodiment, the method for preparing a cosmetic composition according to the invention is characterized in that the mass percentage of the antioxidant is at least 0.01% relative to the total mass of the cosmetic composition.

In one embodiment, the method for preparing a cosmetic composition according to the invention is characterized in that the mass percentage of the antioxidant is at least 0.1% relative to the total mass of the cosmetic composition.

In one embodiment, the method for preparing a cosmetic composition according to the invention is characterized in that the mass percentage of the antioxidant is between 0.01% and 2% relative to the total mass of the cosmetic composition.

In one embodiment, the method for preparing a cosmetic composition according to the invention is characterized in that the mass percentage of the antioxidant is between 0.01% and 1% relative to the total mass of the cosmetic composition.

In one embodiment, the method for preparing a cosmetic composition according to the invention is characterized in that the mass percentage of the antioxidant is between 0.1% and 1% relative to the total mass of the cosmetic composition.

In one embodiment, the method for preparing a cosmetic composition according to the invention is characterized in that the mass percentage of the antioxidant is between 0.01% and 0.1% relative to the total mass of the cosmetic composition.

In one embodiment, the process for preparing a cosmetic composition according to the invention is characterized in that it further comprises a step of mixing a volatile mixture of alkanes obtained by the process according to the invention with at least one pigmentary part.

This pigmentary part allows to obtain a coloring of the cosmetic composition. The pigments used are of synthetic, mineral or vegetable origin. Their dosage is also subtle.

In one embodiment, the method for preparing a cosmetic composition according to the invention is characterized in that the pigmentary part is chosen from the group comprising: water-soluble dyes, liposoluble dyes, pigments, mother-of-pearl, glitter and mixtures thereof.

In one embodiment, the method for preparing a cosmetic composition according to the invention is characterized in that the pigmentary part is chosen from the group comprising: organic pigments such as fixed pigments or precipitated pigments, mineral pigments such as titanium dioxide or iron oxides, sodium aluminosilicate thiosulfates, chromium oxides, bismuth oxychloride, copper phthalocyanine, metal-free phthalocyanine, chlorinated copper phthalocyanine.

Thus, the titanium oxide amount determines the coverage and opacity of the film.

The use of lacquers or precipitated pigments determines the color and chrominance.

For illustration purposes, other types of pigments can be used such as titanium mica, borosilicates and their derivatives, mica, silica.

Mineral pigments are used to nuance the hues while compounds such as bismuth oxychloride can be used to impart particular effects (metallic, shimmering effects, etc.).

In one embodiment, the method for preparing a cosmetic composition according to the invention is characterized in that the pigmentary part comprises a synthetic dye selected from the group comprising: CI100006, CI12010, CI12085, CI12120, CI12370, CI12420, CI12490, CI14270, CI14700, CI14720, CI14815, CI15525, CI15580, CI15620, CI15630, CI15850, CI15865, CI15880, CI15980, CI15985, CI1035, CI16255, CI16290, CI17200, CI18050, CI45100, CI45220, CI45380, CI45430.

In one embodiment, the process for preparing a cosmetic composition according to the invention is characterized in that the pigmentary part comprises a synthetic dye chosen from the group comprising: CI77499, CI77267, CI77268, CI77499 CI77266, CI50420, CI27755, CI28440, CI20470 and mixtures thereof.

In one embodiment, the method for preparing a cosmetic composition according to the invention is characterized in that the pigmentary part comprises a dye of plant origin chosen from fruit and/or plant extracts containing dyes.

In one embodiment, the process for preparing a cosmetic composition according to the invention is characterized in that the mass percentage of the pigmentary part is at least 0.01% relative to the total mass of the cosmetic composition.

In one embodiment, the process for preparing a cosmetic composition according to the invention is characterized in that the mass percentage of the pigmentary part is at least 1% relative to the total mass of the cosmetic composition.

In one embodiment, the process for preparing a cosmetic composition according to the invention is characterized in that the mass percentage of the pigmentary part is at least 5% relative to the total mass of the cosmetic composition.

In one embodiment, the method for preparing a cosmetic composition according to the invention is characterized in that the mass percentage of the pigmentary part is between 0.01% and 20% relative to the total mass of the cosmetic composition.

In one embodiment, the process for preparing a cosmetic composition according to the invention is characterized in that the mass percentage of the pigmentary part is between 1% and 20% relative to the total mass of the cosmetic composition.

In one embodiment, the process for preparing a cosmetic composition according to the invention is characterized in that the mass percentage of the pigmentary part is between 5% and 20% relative to the total mass of the cosmetic composition.

In one embodiment, the process for preparing a cosmetic composition according to the invention is characterized in that the mass percentage of the pigmentary part is between 1% and 15% relative to the total mass of the cosmetic composition.

In one embodiment, the process for preparing a cosmetic composition according to the invention is characterized in that the mass percentage of the pigmentary part is between 5% and 15% relative to the total mass of the cosmetic composition.

In one embodiment, the process for preparing a cosmetic composition according to the invention is characterized in that the mass percentage of the pigmentary part is between 0.01% and 5% relative to the total mass of the cosmetic composition.

In one embodiment, the process for preparing a cosmetic composition according to the invention is characterized in that the mass percentage of the pigmentary part is between 5% and 10% relative to the total mass of the cosmetic composition.

In one embodiment, the process for preparing a cosmetic composition according to the invention is characterized in that the mass percentage of the pigmentary part is between 10% and 15% relative to the total mass of the cosmetic composition.

In one embodiment, the process for preparing a cosmetic composition according to the invention is characterized in that it further comprises a step of mixing a volatile mixture of alkanes obtained by the process according to the invention with at least one surfactant.

For the purposes of the present invention, surfactant is understood to mean a compound which modifies the surface tension between two surfaces, phases. Generally speaking, these compounds are amphiphilic molecules and make it possible to solubilize two immiscible phases of a composition.

In I one embodiment, the process for preparing a cosmetic composition according to the invention is characterized in that the at least one surfactant is chosen from the group comprising nonionic, anionic and amphoteric surfactants.

In one embodiment, the process for preparing a cosmetic composition according to the invention is characterized in that the at least one surfactant is a non-ionic surfactant.

In one embodiment, the method for preparing a cosmetic composition according to the invention is characterized in that the at least one surfactant is chosen from the group of nonionic surfactants consisting of: polyoxyalkylene alkyl ethers, glycerin alkyl ethers, glycerin fatty acid esters, polyglycerin fatty acid esters, sorbitan fatty acid esters, their derivatives, and their mixtures.

In one embodiment, the process for preparing a cosmetic composition according to the invention is characterized in that the at least one surfactant is an anionic surfactant.

In one embodiment, the process for preparing a cosmetic composition according to the invention is characterized in that the at least one surfactant is an anionic surfactant chosen from the group consisting of: alkyl phosphates, polyoxyalkylene alkyl ether phosphates, sulfonates, alkyl sulfates, polyaspartates, their derivatives and their mixtures.

In one embodiment, the process for preparing a cosmetic composition according to the invention is characterized in that the at least one surfactant is an amphoteric surfactant.

In one embodiment, the process for preparing a cosmetic composition according to the invention is characterized in that the mass percentage of the at least surfactant is at least 0.1% relative to the total mass of the cosmetic composition.

In one embodiment, the process for preparing a cosmetic composition according to the invention is characterized in that the mass percentage of the at least surfactant is at least 5% relative to the total mass of the cosmetic composition.

In one embodiment, the process for preparing a cosmetic composition according to the invention is characterized in that the mass percentage of the at least one surfactant is between 0.1% and 30% relative to the total mass of the cosmetic composition.

In one embodiment, the process for preparing a cosmetic composition according to the invention is characterized in that the mass percentage of the at least one surfactant is between 5% and 15% relative to the total mass of the cosmetic composition.

In one embodiment, the process for preparing a cosmetic composition according to the invention is characterized in that it further comprises a step of mixing a volatile mixture of alkanes obtained by the process according to the invention with at least one aqueous phase.

In one embodiment, the process for preparing a cosmetic composition according to the invention is characterized in that the aqueous phase consists of water.

In one embodiment, the process for preparing a cosmetic composition according to the invention is characterized in that the aqueous phase comprises water and at least one water-miscible organic solvent.

In one embodiment, the process for preparing a cosmetic composition according to the invention is characterized in that the aqueous phase comprises at least one water-miscible organic solvent chosen from the group consisting of lower alcohols such as ethanol, isopropanol, butanol, isoamyl alcohol, glycols such as ethylene glycol, propylene glycol, 1,3-butylene glycol, ketones and short-chain aldehydes in C₂ to C₄.

In one embodiment, the process for preparing a cosmetic composition according to the invention is characterized in that the mass percentage of the aqueous phase is between 1 and 95% relative to the total mass of the cosmetic composition.

In one embodiment, the process for preparing a cosmetic composition according to the invention is characterized in that the mass percentage of the aqueous phase is between 5 and 80% relative to the total mass of the cosmetic composition.

In one embodiment, the process for preparing a cosmetic composition according to the invention is characterized in that the mass percentage of the aqueous phase is between 5 and 60% relative to the total mass of the cosmetic composition.

In one embodiment, the process for preparing a cosmetic composition according to the invention is characterized in that the mass percentage of the aqueous phase is between 1 and 50% relative to the total mass of the cosmetic composition.

In one embodiment, the process for preparing a cosmetic composition according to the invention is characterized in that the mass percentage of the aqueous phase is between 1 and 30% relative to the total mass of the cosmetic composition.

In one embodiment, the process for preparing a cosmetic composition according to the invention is characterized in that the mass percentage of the aqueous phase is between 10 and 40% relative to the total mass of the cosmetic composition.

In one embodiment, the process for preparing a cosmetic composition according to the invention is characterized in that the mass percentage of the aqueous phase is between 1 and 20% relative to the total mass of the cosmetic composition.

In one embodiment, the method for preparing a cosmetic composition according to the invention is characterized in that the cosmetic composition obtained has a naturalness index greater than or equal to 80% carbon 14 (¹⁴C) determined according to one of the following standards ASTM D6866, EN 16640 or EN 16785-1.

In one embodiment, the method for preparing a cosmetic composition according to the invention is characterized in that the cosmetic composition obtained has a naturalness index greater than or equal to 85% carbon 14 (¹⁴C) determined according to one of the following standards ASTM D6866, EN 16640 or EN 16785-1.

In one embodiment, the process for preparing a cosmetic composition

according to the invention is characterized in that the cosmetic composition obtained has a naturalness index greater than or equal to 90% carbon 14 (¹⁴C) determined according to one of the following standards ASTM D6866, EN 16640 or EN 16785-1.

In one embodiment, the method for preparing a cosmetic composition according to the invention is characterized in that the cosmetic composition obtained has a naturalness index greater than or equal to 95% carbon 14 (¹⁴C) determined according to one of the following standards ASTM D6866, EN 16640 or EN 16785-1.

In one embodiment, the process for preparing a cosmetic composition according to the invention is characterized in that at least 85% by mass of its ingredients is biosourced relative to the total mass of the composition.

In one embodiment, the method for preparing a cosmetic composition according to the invention is characterized in that at least 50% by mass of the ingredients of the cosmetic composition obtained is of natural origin relative to the total mass of the composition.

In one embodiment, the method for preparing a cosmetic composition according to the invention is characterized in that at least 75% by mass of the ingredients of the cosmetic composition obtained is of natural origin relative to the total mass of the composition.

In one embodiment, the method for preparing a cosmetic composition according to the invention is characterized in that at least 90% by mass of the ingredients of the cosmetic composition obtained is of natural origin relative to the total mass of the composition.

In one embodiment, the method for preparing a cosmetic composition according to the invention is characterized in that 50% by mass of the ingredients of the cosmetic composition obtained is of natural origin relative to the total mass of the composition.

In one embodiment, the method for preparing a cosmetic composition according to the invention is characterized in that 75% by mass of the ingredients of the cosmetic composition obtained is of natural origin relative to the total mass of the composition.

In one embodiment, the method for preparing a cosmetic composition according to the invention is characterized in that 90% by mass of the ingredients of the cosmetic composition obtained is of natural origin relative to the total mass of the composition.

The invention also relates to the use of the volatile mixture of alkanes obtained by the process according to the invention for the preparation of a cosmetic composition for lipstick, long-lasting lipstick or lip gloss.

For the purposes of the present invention, the term “long-lasting” lipstick, also known as “non-transfer” lipstick, means a lipstick that does not leave marks, is water-resistant and sometimes oil-resistant.

Similarly, “lip gloss” or “gloss” is a makeup product that gives a shiny and/or wet look to the lips, and sometimes, to color or even glitter them. Some of these lip glosses are transparent and can be applied over a classic lipstick to add a shiny effect.

Traditionally, according to the book Conception des produits cosmétiques: la formulation, by Anne-Marie PENSE-LHERITIER published in 2014 by LAVOISIER, the composition of a lipstick is structured around a pair:
Base or support
Pigmentary part

The base contains different types of ingredients selected mainly on their organoleptic properties:
volatile solvents to facilitate application and hold;
oils to provide slip and ease of application;
waxes, to fix consistency and hardness;
fatty substances such as pasty fats or gelling agents to adjust the smoothness and ease of use.

Obtaining a lipstick is done by finding a subtle balance between these different ingredients.

These ingredients can be supplemented with various additives providing specific properties: polymers, absorbent powders, stabilizing additives (antioxidants, preservatives, etc.), perfume and active ingredients where applicable.

In one embodiment, the cosmetic compositions for long-lasting lipstick, or lip glosses comprising the volatile mixture of alkanes obtained by the process according to the invention have excellent stability and remarkable cosmetic qualities such as ease of application, water resistance, good hold, more intense and luminous color.

The invention also relates to the use of the volatile mixture of alkanes obtained by the process according to the invention for the preparation of a cosmetic eye makeup composition.

In one embodiment, the volatile mixture of alkanes obtained by the process according to the invention is used for the preparation of a cosmetic composition of mascara or eyeliner.

Traditionally, according to the book Conception des produits cosmétiques: la formulation, by Anne-Marie PENSE-LHERITIER published in 2014 by LAVOISIER, there are mainly two types of mascara formulations:
Continuous water-phase mascaras are also called “water-based mascara”
Continuous oil phase mascaras are also called “waterproof mascara”

In one embodiment, the volatile mixture of alkanes obtained by the process according to the invention is used for the preparation of cosmetic compositions of mascaras with a continuous oily phase.

Continuous oil phase mascaras are mainly based on the use of wax or continuous oil phase emulsion. The aim is to form a continuous film on the surface of the eyelash, a film that is perfectly water-resistant.

To do this, the mascara composition includes in particular an oily phase and a pigmentary part and this composition may optionally include an aqueous phase.

In one embodiment, the mascara composition comprising the volatile mixture of alkanes obtained by the process according to the invention comprises a mass percentage of less than or equal to 10% of water relative to the total mass of the composition.

In a preferred embodiment, the mascara composition comprising the volatile mixture of alkanes obtained by the process according to the invention is free of an aqueous phase.

The fatty phase includes in particular, volatile solvents, oils, surfactants, waxes, gelling agents, film-forming polymers, and preservatives.

The pigment part includes water-soluble dyes, fat-soluble dyes, pigments, mother of pearls and glitters.

Obtaining a mascara is done by finding a subtle balance between these different ingredients.

To these ingredients various additives can be added providing specific properties: polymers, absorbent powders, stabilizing additives such as antioxidants, perfume and active ingredients where appropriate.

Regarding eyeliners, their composition is similar to that of the mascaras developed above.

In one embodiment, the cosmetic compositions for the eyes, such as mascara or eyeliner, comprising the volatile mixture of alkanes obtained by the process according to the invention have excellent stability and remarkable cosmetic qualities such as ease of application, water resistance, good hold and a more intense color.

The invention also relates to the use of the volatile mixture of alkanes obtained by the process according to the invention for the preparation of a cosmetic composition applicable to the skin.

In one embodiment, the volatile mixture of alkanes obtained by the process according to the invention is particularly used for the preparation of cosmetic compositions such as a makeup primer or base, a blush, an eye shadow, a foundation or a makeup remover formulation.

Foundation is a formulation intended to unify the skin and lightly color it. It must be applied in a uniform layer but also allow blending, on the face and part of the neck.

Similarly, we understand by base, primer or makeup base a cosmetic composition that allows to blur skin imperfections and prevents makeup from settling in wrinkles, pores throughout the day. Thus, this formulation allows to perfect the skin before applying a foundation, a blush, etc.

Rouge make-up, also commonly called “blush”, is a cosmetic formulation intended to mimic or reinforce a “rosy cheek” appearance.

Similarly, eyelids make-up or commonly called “eye-shadow” means cosmetic formulations applicable to the eyelid in order to highlight the eyes by creating color contrasts.

By makeup remover formulation we mean a makeup remover oil but also a biphasic makeup remover formulation which allows the face to be cleansed of makeup while respecting the skin of its user.

As stated above, these makeup remover formulations can come in the form of a makeup remover oil made up of oils and various fatty substances.

They can also be presented in the form of a biphasic formulation comprising an aqueous phase and an oily phase.

Conventionally, the volatile mixture of alkanes obtained by the process according to the invention is used for the preparation of cosmetic compositions applicable to the skin, in particular foundation and/or base compositions which can be formulated in a fluid or solid form of powder type, loose compact or slur.

These compositions may in particular, independently of one another, be presented in an anhydrous form or in the form of an emulsion.

These emulsions can come in different forms, namely in the form of an oil-in-water emulsion, water-in-oil, but also under certain conditions in the form of a multiple W/O/W (water/oil/water) or even O/W/O (oil/water/oil) emulsion combining at least one aqueous phase and one fatty phase.

In one embodiment, the volatile mixture of alkanes obtained by the process according to the invention is used for the preparation of a cosmetic composition applicable to the skin comprising at least one aqueous phase and one fatty phase.

The aqueous phase consists mainly of water.

The fatty phase contains different types of ingredients selected mainly on their organoleptic properties:
volatile solvents to facilitate application and hold
oils to provide slip and ease of application
waxes, to fix consistency and hardness
fatty substances such as pasty fats or gelling agents to adjust the smoothness and ease of use.

Generally, the volatile mixture of alkanes obtained by the process according to the invention is used for the preparation of a cosmetic composition applicable to the skin comprising a pigmentary part.

Obtaining a cosmetic composition that is applicable to the skin is achieved by finding a subtle balance between these different ingredients.

These ingredients can be supplemented with various additives providing specific properties: polymers, absorbent powders, stabilizing additives (antioxidants, preservatives, etc.), perfume and active ingredients where applicable.

In one embodiment, the cosmetic compositions applicable to the skin such as bases or primers, foundations or powders, comprising the volatile mixture of alkanes obtained by the process according to the invention have excellent stability and remarkable cosmetic properties such as ease of application, water resistance, good hold, more intense and luminous color.

The invention also relates to the use of the volatile mixture of alkanes obtained by the process according to the invention for the preparation of a cosmetic composition for the treatment of keratin fibers, in particular hair.

In one embodiment, the volatile mixture of alkanes obtained by the process according to the invention is used for the preparation of cosmetic compositions for the treatment of keratin fibers in the form of a solution, dispersion, oil/water emulsion, water/oil emulsion, water/oil/water emulsion, powder, talc, capsules, sponge, mousse, lacquer and gel.

In one embodiment, the volatile mixture of alkanes obtained by the process according to the invention is used for the preparation of cosmetic compositions for the treatment of keratin fibers such as hair toners, hair dyes, hair styling products, shampoos, hair conditioners, conditioners, conditioning shampoos, hair treatment products, hair sprays, hair masks, hair care products, hair treatment products, permanent wave fixing solutions, hair shaping products, shampoos for colored hair, hair fixatives, hair holding products, hair styling preparations, leave-in hair products, curling iron lotions, fixing mousses, hair gels, hair waxes or combinations thereof.

In one embodiment, the cosmetic compositions for the treatment of keratin fibers, in particular hair, comprising the volatile mixture of alkanes obtained by the process according to the invention exhibit excellent stability and remarkable cosmetic properties such as ease of application, good hold, luminous color and more intense shine.

### EXAMPLES

The following examples are given to illustrate the invention.

Gas chromatography method used for the determination of purity

The purity of the compounds was determined by gas chromatography (GC) using a GC FID 8860 (Agilent) equipped with a ZB5HT Inferno column (Phenomenex) with dimensions of 15 m×0.32 mm×0.10 mm, a split injector heated to 325° C. and a FID detector heated to 350° C.

The sample to be analyzed was prepared by a standard silylation method before injection (1 μL) using the BSTFA/TMCS derivatization mixture (Sigma Aldrich).

The injection was carried out between 300 and 330° C. depending on the sample type and was followed by a heating gradient between 100 and 400° C.

Squalane was used as an internal standard for the analysis of Guerbet alcohols while 1-hexanol was used as an internal standard for the analysis of alkene mixtures and volatile alkane mixtures.

### Method for Determining the Iodine Index

The iodine index of the volatile mixtures of alkanes obtained by the process according to the invention was determined according to the NF EN ISO 3961 method.

### Example 1: Process for the Preparation of a Volatile Mixture of 2,3,6-trimethylheptane and n-dodecane According to the Invention

### Step 1: Synthesis of 2-isopropyl-5-methylhexan-1-ol from Isoamyl Alcohol (CAS 123-51-3) by Guerbet Synthesis

Isoamyl alcohol (Sigma Aldrich) has a minimum mass purity of 85% in 3-methylbutanol.

The synthesis is carried out under the following conditions:

Reagents used:

TABLE 1

| | | |
|---|---|---|
| Alcohol isoamyl (3-methylbutanol ≥85%) | 75.8 | g |
| Mass | | |
| Base: KOH 85% | 1.1 | g |
| Mass | | |
| Catalyst: Pricat Cu 50/8 | 19.0 | mg |
| Mass | | |

Operating conditions:

TABLE 2

| Temperature | 180 to 230° | C. |
|---|---|---|
| Duration | 24 | hours |

Composition of the crude product obtained by the Guerbet synthesis (CPG analysis):

TABLE 3

| | |
|---|---|
| Isoamyl alcohol | <5% |
| 2-isopropyl-5-methylhexan-1-ol | >75% |
| Heavy products | ≤20% |

Heavy products include compounds resulting from trimerization of the reactive alcohol.

The crude product is purified by vacuum distillation.

Topping allows the elimination of traces of isoamyl alcohol.

Tailing then allows the elimination of heavy products.

The Guerbet alcohol 2-isopropyl-5-methylhexan-1-ol obtained has a purity greater than 94% by CPG analysis.

### Step 2

70 g of bio-based 1-dodecanol (purity 99%, BASF) are added to 30 g of Guerbet alcohol 2-isopropyl-5-methylhexan-1-ol to form a mixture of bio-based alcohols.

### Step 3

### Dehydration of the Alcohol Mixture 1-dodecanol/2-isopropyl-5-methylhexan-1-ol

A catalyst bed is placed at mid-height of the reactor containing the alcohol mixture. A thermocouple is placed at the heart of this bed. The latter is held by a grid on which silica wool (VWR) is placed. Above the bed, silica wool is added in order to block the alumina catalyst bed (Johnson Matthey).

The LHSV (Liquid Hourly Space Velocity) corresponds to the flow rate of alcohol passed through the reactor expressed in mL/min/mL of catalyst.

characteristic parameters of the continuous dehydration process are as follows:

TABLE 4

| Temperature | LHSV | Rate |
|---|---|---|
| 330° C. | 2.5 | 0.5 mL/min |

The resulting mixture of alkenes is a liquid with a purity greater than 99.5% (CPG analysis).

### Hydrogenation of the Mixture of Alkenes Obtained During the Dehydration Reaction

The hydrogenation reaction takes place in a batch reactor with a capacity of one liter.

Mixture of alkenes (100 g) and Raney nickel catalyst of the Nysosel type (BASF) (0.5 g) are introduced into the reactor at room temperature. The reactor is inerted with 3×5 bars of nitrogen and the temperature is raised to a temperature of 180° C. 5 bars of hydrogen are then introduced. After 2 hours, the temperature is raised to 200° C. and the hydrogen pressure to 10 bars. The reaction is then continued for a further 3 hours.

A volatile alkane mixture consisting of 2,3,6-trimethylheptane and n-dodecane is obtained. The composition of the volatile alkane mixture is 30% by mass of 2,3,6-trimethylheptane and 70% by mass of n-dodecane.

The volatile alkane mixture has a purity of 99.7% (CPG analysis), is odorless and the iodine value of the volatile alkane mixture is less than 1 g of iodine per 100 g of mixture.

### Example 2: Process for the Preparation of a Volatile Mixture of 5-methylundecane and n-dodecane According to the Invention

### Step 1: Synthesis of 2-butyloctan-1-ol from Plant 1-hexanol by Guerbet Synthesis

Vegetable 1-hexanol (Sigma Aldrich) has a mass purity of 99%.

The synthesis is carried out under the following conditions:

Reagents used:

TABLE 5

| | | |
|---|---|---|
| 1-hexanol 99% purity | 87.7 | g |
| Mass | | |
| Base: KOH 85% | 1.3 | g |
| Mass | | |
| Catalyst: Pricat Cu 50/8 | 22.0 | mg |
| Mass | | |

Operating conditions:

TABLE 6

| Temperature | 180 to 230° | C. |
|---|---|---|
| Duration | 24 | hours |

Composition of the crude product obtained by the Guerbet synthesis (analysis by GC):

TABLE 7

| | |
|---|---|
| 1-hexanol | <5% |
| 2-butyloctan-1-ol | >75% |
| Heavy products | ≤20% |

Heavy products include compounds resulting from trimerization of 1-hexanol.

The crude product is purified by vacuum distillation.

Topping allows the elimination of traces of 1-hexanol.

Tailing then allows the elimination of heavy products.

The Guerbet alcohol 2-butyloctan-1-ol obtained has a purity greater than 94% by CPG analysis.

### Step 2

50 g of bio-based 1-dodecanol (purity 99%, BASF) are added to 50 g of Guerbet alcohol 2-butyloctan-1-ol to form a mixture of bio-based alcohols.

### Step 3

### Dehydration of the Alcohol Mixture 1-dodecanol/2-butyloctan-1-ol

The dehydration reaction of the 1-dodecanol/2-butyloctanol alcohol mixture of step 2 is carried out by a method similar to that described in step 3 of example 1.

The resulting mixture of alkenes is a liquid with a purity greater than 99.5% (CPG analysis).

### Hydrogenation of the Mixture of Alkenes Obtained During the Dehydration Reaction

By a process similar to that described for step 3 of example 1, the mixture of alkenes (100 g) obtained by dehydration is hydrogenated in the presence of a Nysosel type Raney nickel catalyst (BASF) (0.5 g).

A volatile mixture of alkanes consisting of 5-methylundecane and n-dodecane is obtained. The composition of the volatile mixture of alkanes is 50% by mass of 5-methylundecane and 50% by mass of n-dodecane.

The volatile alkane mixture has a purity of 99.7% (CPG analysis), is odorless and the iodine value of the volatile alkane mixture is less than 1 g of iodine per 100 g of mixture.

### Example 3: Process for the Preparation of a Volatile Mixture of 2,3,6-trimethylheptane, 5-methylundecane and n-dodecane According to the Invention

### Step 1

Guerbet alcohol 2-isopropyl-5-methylhexan-1-ol is obtained by the process described in Example 1 from isoamyl alcohol.

Guerbet alcohol 2-butyloctan-1-ol is obtained by the process described in Example 2 from 1-hexanol.

### Step 2

40 g of bio-based 1-dodecanol (purity 99%, BASF) are added to 30 g of 2-butyloctan-1-ol and 30 g of 2-isopropyl-5-methylhexan-1-ol to form a mixture of bio-based alcohols.

### Step 3

### Dehydration of the Alcohol Mixture 1-dodecanol/2-butyloctan-1-ol/2-isopropyl-5-methylhexan-1-ol

The dehydration reaction of the alcohol mixture 1-dodecanol/2-butyloctan-1-ol/2-isopropyl-5-methylhexan-1-ol of step 2 is carried out by a method similar to that described in step 3 of example 1.

The resulting mixture of alkenes is a liquid with a purity greater than 99.5% (CPG analysis).

### Hydrogenation of the Mixture of Alkenes Obtained During the Dehydration Reaction

By a process similar to that described in step 3 of example 1, the mixture of alkenes (100 g) obtained by dehydration is hydrogenated in the presence of a Nysosel type Raney nickel catalyst (BASF) (0.5 g).

A volatile alkane mixture consisting of 2,3,6-trimethylheptane, 5-methylundecane and n-dodecane is obtained. The composition of the volatile alkane mixture is 30% by mass of 2,3,6-trimethylheptane, 30% of 5-methylundecane and 40% by mass of n-dodecane.

The volatile alkane mixture has a purity of 99.7% (GC analysis), is odorless and the iodine value of the volatile alkane mixture is less than 1 g of iodine per 100 g of mixture.

### Example 4: Preparation of a Long-Lasting Lipstick Cosmetic Composition

A long-lasting lipstick cosmetic composition is prepared from the volatile mixture of alkanes prepared in Example 1.

The detailed composition of this long-lasting lipstick formulation is given in Table 8 below:

Cosmetic composition of long-lasting lipstick

| Phase | Trade Name | INCI | % |
|---|---|---|---|
| 1 | Volatile mixture of alkanes | | 34.05 |
| | (2,3,6-trimethylheptane/n-dodecane) | | |
| | AEROSIL | SILICA | 1.00 |
| 2 | C47-5001 SUNPURO | TITANIUM DIOXIDE | 0.56 |
| | C37038 SUNCROMA D&C | CI 73360 (AND) ALUMINUM | 4.39 |
| | RED 30 AI | HYDROXIDE | |
| | C19011 SUNCROMA DC | CI 15850 (AND) RED 7 LAKE | 2.20 |
| | RED 7 CA LAKE | | |
| | C694424 SUNCROMA FDC | CI 19140 | 3.69 |
| | YELLOW 5 AL LAKE | | |
| | C339001 SUNPRO | CI 77492 | 1.64 |
| | YELLOW IRON OXIDE | | |
| | TMS 803 | TRIMETHYLSILLOXYSILICATE | 13.00 |
| | OLIVE GEL SQE | Dimer dilinoleyl dilinoleate & olive | 8.53 |
| | | (olea europaea) oil | |
| | | unsaponifiables & dilinoleic acid/ | |
| | | butanediol copolymer | |
| | BENTONE GEL ISD V | ISODODECANE (AND) | 4.11 |
| | | DISTEARDIMONIUM HECTORITE | |
| | | (AND) PROPYLENE CARBONATE | |
| 3 | ARLAMOL HD | ISOHEXADECANE | 6.85 |
| | RHEOPEARL KL2 | DEXTRIN PALMITATE | 5.48 |
| 4 | POLYBUTENE | POLYBUTENE | 4.50 |
| | ANTARON V 220 | VP/EICOSENE COPOLYMER | 3.00 |
| | CEROZO RS/LA C | OZOKERITE | 5.00 |
| | MIYOSYN MATTE ALT | SYNTHETIC FLUORPHLOGOPITE | 2.00 |
| TOTAL | | | 100.00 |

In order to prepare the long-lasting lipstick cosmetic composition, AEROSIL is dispersed in the volatile alkane mixture to form phase 1 of the lipstick formulation.

Once phase 1 has dispersed, phase 2 is weighed in another container at the same time. The same operation is carried out for phase 3 and then phases 2 and 3 are mixed under magnetic stirring.

The mixture of phases 2 and 3 is then placed on a hot plate until the RHEOPEARL KL2 is completely dispersed. The dispersed phase 1 is then added to the mixture of phases 2 and 3.

Finally, phase 4 is heated alone on a hot plate until the wax has completely melted, then phase 4 is cooled in a cold water bath at 40° C. and added to the mixture of dispersed phases 1, 2 and 3.

This gives a homogeneous cosmetic composition of long-lasting lipstick.

### Example 5: Preparation of a Cosmetic Lip Gloss Composition

A cosmetic lip gloss composition is prepared from a volatile mixture of alkanes consisting of 20% by mass of 2,3,6-trimethylheptane and 80% by mass of n-dodecane. This volatile mixture of alkanes is prepared by a process similar to that described in Example 1.

The detailed composition of this lip gloss formulation is given in Table 10 below:

Cosmetic composition of lip gloss

| Phase | Trade name | INCI | % |
|---|---|---|---|
| 1 | Volatile mixture of alkanes | | 5.00 |
| | (2,3,6-trimethylheptane/n-dodecane) & | | |
| | STEARALKONIUM BENTONITE & TRIETHYL CITRATE | | |
| | SUNPURO RED | CI 77491 | 0.10 |
| | SUNPURO YELLOW | CI 77492 | 0.16 |
| | SUNPURO TiO2 | CI 15850 | 0.23 |
| | C19011 SUNCROMA DC | CI 77891 | 0.15 |
| | RED 7 CA LAKE | | |
| | EUTANOL G | OCTYLDODECANOL | 13.36 |
| 2 | SWEET ALMOND OIL | Prunus Amygdalus Dulcis (Sweet | 21.00 |
| | | Almond) Oil | |
| | CRODAMOL PTIS | PENTAERYTHRITYL TETRAISOSTEARATE | 10.00 |
| | KAHLWAX 6607 H | HELIANTHUS ANNUUS SEED WAX | 5.00 |
| | LUSPLAN DD 07 | DIMER DILINOLEYL DIMER DILINOLEATE | 35.00 |
| | VISCOPLAST WR | DILINOLEIC ACID/BUTANEDIOL | 10.00 |
| | | COPOLYMER & C18-21 ALKANE | |

| | |
|---|---|
| TOTAL | 100 |

In order to prepare the cosmetic lip gloss composition, the pigments of phase 1 are mixed with octyldodecanol to obtain a paste. Then, the bentone gel obtained by the combination of the volatile mixture of alkanes obtained by the process according to the invention, a stearalkonium bentonite and triethyl citrate is added. Finally, the entire phase 1 is dispersed.

In this example, the volatile mixture of alkanes constitutes more than 80% by mass relative to the total mass of the bentone gel composition.

In a second step, phase 2 is placed in a water bath at a temperature of 80° C. until the waxes are in a liquid state.

Phase 2 is allowed to cool to 40° C. then mixed with phase 1.

The cosmetic composition of lip gloss has a satisfying glossy effect.

### Example 6: preparation of a Cosmetic Mascara Composition

A cosmetic mascara composition is prepared from a volatile mixture of alkanes consisting of 20% by mass of 2,3,6-trimethylheptane and 80% by mass of n-dodecane. This volatile mixture of alkanes is prepared by a process similar to that described in Example 1.

The detailed composition of this mascara formulation is given in Table 10 below:

Cosmetic composition of mascara

| Phase | Trade name | INCI | % by mass |
|---|---|---|---|
| 1 | Volatile mixture of alkanes | | 25.3 |
| | (2,3,6-trimethylheptane/n-dodecane) | | |
| | VEGELIGHT BENTONE SILK SB | COCONUT ALKANES (AND) | 10 |
| | | DISTEARDIMONIUM HECTORITE | |
| | | (AND) TRIETHYL CITRATE | |
| | TMS 803 | TRIMETHYLSILOXYSILICATE | 1 |
| | MI BLACK | ALUMINUM DIMYRISTATE CI 77499 | 14 |
| | MASSOCARE PEG30 PHS | PEG-30 DIPOLYHYDROXYSTEARATE | 1 |
| | OLIVE GEL SQE | DIMER DILINOLEYL DIMER | 5.0 |
| | | DILINOLEATE & | |
| | | OLIVE (OLEA EUROPAEA) OIL | |
| | | UNSAPONIFIABLES &DILINOLEIC | |
| | | ACID/BUTANEDIOL COPOLYMER | |
| 2 | ANTARON V216 (GANEX) | PVP/HEXADECENE COPOLYMER | 3.0 |
| | KAHLRESIN 6723 | SHOREA ROBUSTA RESIN & | 1.5 |
| | | OCTYLODECANOL | |
| | MYRITOL 318 | CAPRYLIC/CAPRIC TRIGLYCERIDE | 1.8 |
| | CEGESOFT HF 52 | HYDROGENATED VEGETABLE OIL | 10.0 |
| | BEESWAX WHITE SP 422P | BEESWAX | 3.0 |
| | CARNAUBA WAX SP 63P | COPERNICIA CERIFERA (CARNAUBA) | 2.5 |
| | | WAX | |
| | CRODABOND CSA LQ | HYDROGENATED CASTOR | 3.0 |
| | | OIL/SEBACIC ACID COPOLYMER | |
| | PARLEAM | HYDROGENATED POLYISOBUTENE | 2.0 |
| 3 | PHENOXETOL ® | PHENOXYETHANOL | 0.7 |
| | DERMOSOFT OCTIOL | CAPRYLYL GLYCOL | 0.5 |
| 4 | DEIONIZED WATER | WATER | 10.0 |
| | POTASSIUM SORBATE | POTASSIUM SORBATE | 0.2 |
| 5 | SILICA BEAD | SB 700 SILICA | 2 |
| | MIYOSYN MATTE-ALT | MICA (AND) ISOPROPYL TITANIUM | 2 |
| | | TRIISOSTEARATE | |
| | SENSOSEL 10 | CELLULOSE | 1 |
| | ESP ECOCEL 10 | CELLULOSE | 0.5 |

| | |
|---|---|
| TOTAL | 100 |

To prepare the cosmetic mascara composition, phase 1 is weighed and heated to a maximum temperature of 35° C.

Separately, phase 2 is weighed into another container and then phase 2 is heated to a temperature of 60° C. Then, phase 2 is added to phase 1, as well as phase 3 in a second step.

Phase 4 is then added with vigorous magnetic stirring to obtain an emulsion.

The texture of the mascara cosmetic composition is correct.

### Example 7: preparation of a Cosmetic Composition of an Eyeliner

A cosmetic composition of an eyeliner is prepared from a volatile mixture of alkanes consisting of 40% by mass of 2,3,6-trimethylheptane and 60% by mass of n-dodecane. This volatile mixture of alkanes is prepared by a process similar to that described in Example 1.

The detailed composition of this eyeliner formulation is given in Table 11 below:

Cosmetic composition of an eyeliner

| Phase | Trade name | INCI | % by mass |
|---|---|---|---|
| 1 | Volatile mixture of alkanes | | 37.8 |
| | (2,3,6-trimethylheptane/n-dodecane) | | |
| | Vegelight Bentone Silk SB | COCONUT ALKANES (AND) | 10 |
| | | STEARALKONIUM BENTONITE | |
| | | (AND) TRIETHYL CITRATE | |
| | MI BLACK | ALUMINUM DIMYRISTATE | 14 |
| | | (AND) CI 77499 | |
| | OLIVE GEL SQE | DIMER DILINOLEYL DIMER | 5.5 |
| | | DILINOLEATE & OLIVE | |
| | | (OLEA EUROPAEA) OIL | |
| | | UNSAPONIFIABLES | |
| | | &DILINOLEIC | |
| | | ACID/BUTANEDIOL | |
| | | COPOLYMER | |
| | BELSIL TMS 803 | TRIMETHYLSILOXYSILICATE | 3 |
| 2 | MYRITOL 318 | CAPRYLIC CAPRIC | 1.8 |
| | | TRIGLYCERIDE | |
| | VEGEWAX 55 SUN | HYDROGENATED SUNFLOWER | 5 |
| | | SEED OIL | |
| | BEESWAX WHITE SP 422P | BEESWAX | 3 |
| | CRODABOND CSA LQ | HYDROGENATED CASTOR | 1.5 |
| | | OIL/SEBACIC ACID | |
| | | COPOLYMER | |
| | Volatile mixture of alkanes | | 2 |
| | according to the present invention | | |
| 3 | MICROCARE PE | PHENOXYETHANOL | 0.2 |
| | DERMOSOFT OCTIOL | CAPRYLYL GLYCOL | 0.5 |
| 4 | DEIONIZED WATER | WATER | 10 |
| | POTASSIUM SORBATE | POTASSIUM SORBATE | 0.2 |
| 5 | SILICA BEAD SB 700 | SB 700 SILICA | 2 |
| | MIYOSYN MATTE-ALT | SYNTHETIC FLUORPHLOGOPITE | 2 |
| | | (AND) | |
| | | TRIETHOXYCAPRYLYLSILANE | |
| | SENSOSEL 10 | CELLULOSE | 1.5 |

| | |
|---|---|
| TOTAL | 100 |

In order to prepare the cosmetic composition of an eyeliner of example 6, phase 1 is weighed and heated to a maximum temperature of 40° C.

Separately, phase 2 is weighed into another container and then phase 2 is heated to a temperature of 60° C. Then, after cooling, phase 2 is added to phase 1, as well as phase 3 in a second step.

Phase 4 is then added gradually with vigorous magnetic stirring to obtain an emulsion.

Finally, add the powders from phase 5 to the mixture.

This cosmetic composition of an eyeliner has a naturalness index equal to 90% according to the ASTM D6866 standard.

In other words, this cosmetic composition is made up of 90% ingredients of natural origin.

### Example 8: Preparation of a Cosmetic Composition of a Foundation

A cosmetic composition of a foundation is prepared from a volatile mixture of alkanes consisting of 45% by mass of 2,3,6-trimethylheptane and 55% by mass of n-dodecane. This volatile mixture of alkanes is prepared by a process similar to that described in Example 1.

The detailed composition of this cosmetic formulation of a foundation is given in Table 12 below:

Cosmetic composition of a foundation

| Phase | Trade name | INCI | % by mass |
|---|---|---|---|
| 1 | Volatile mixture of alkanes | | 25.74 |
| | (2,3,6-trimethylheptane/n-dodecane) | | |
| | MYRITOL 318 | Caprylic/Capric Triglyceride | 2.75 |
| 2 | ALT TAO 77891 | CI 77891 & | 8.58 |
| | | TRIETHOXCAPRYLYLSILANE | |
| | ALT C339001-10 | CI 77492 & | 1.84 |
| | | TRIETHOXCAPRYLYLSILANE | |
| | ALT C338001-10 | CI 77491 & | 0.46 |
| | | TRIETHOXCAPRYLYLSILANE | |
| | ALT C337001-10 | CI 77499 & | 0.14 |
| | | TRIETHOXCAPRYLYLSILANE | |
| 3 | SR1000 MQ RESIN | TRIMETHYLSILOXYSILICATE | 1.93 |
| 4 | DERMOSOFT | GLYCERYL CAPRYLATE | 0.49 |
| | GMCY | | |
| | VEGELIGHT SI- | COCONUT ALKANES & DIMETHICONE | 9.69 |
| | 118 | CROSSPOLYMER | |
| | KF-56A | PHENYL TRIMETHICONE | 2.9 |
| | KF 6017 | PEG-10 DIMETHICONE | 5.61 |
| | SPAN 83 LQMV | SORBITAN SESQUIOLEATE | 0.1 |
| | BENTONE GEL ISD | ISODODECANE & DISTEARDIMONIUM | 6.13 |
| | V | HECTORITE & PROPYLENE | |
| | | CARBONATE | |
| | MICROCARE PE | PHENOXYETHANOL | 0.37 |
| | MLW EXCURSION | SCENT | 0.02 |
| 5 | OSMOSIS WATER | AQUA | 28.05 |
| | HYDROLITE-5 | PENTYLENE GLYCOL | 0.19 |
| | CHLORPHENESIN | CHLORPHENESIN | 0.19 |
| | SODIUM | SODIUM CHLORIDE | 0.49 |
| | CHLORIDE | | |
| | MAGNESIUM | MAGNESIUM SULFATE | 0.49 |
| | SULFATE | | |
| | HEPTAHYDRATE | | |
| 6 | ZEMEA | PROPANEDIOL | 1.4 |
| | PROPANEDIOL | | |
| | KELTROL TF | XANTHAN GUM | 0.25 |
| 7 | TALC P3 | TALC | 2.19 |

| | |
|---|---|
| TOTAL | 100 |

In order to prepare the cosmetic composition of a foundation of example 7, phase 2 is dispersed in phase 1 with progressive stirring in the Turrax™ up to 12000 rpm for 2 minutes. Then, phase 3 is added to phases 1 and 2. Finally, phase 4 is added to the mixture of phases 1, 2 and 3.

The mixture of phases 1, 2, 3 and 4 constitutes the oily phase of the cosmetic composition.

Phases 5 and 6 (aqueous phase) are then added to the mixture of phases 1, 2, 3 and 4 to make the emulsion. The mixture obtained is gradually stirred with the Turrax™ up to 12000 rpm for 2 minutes.

Finally, phase 7 is dispersed in the emulsion by progressive stirring with the Turrax™ up to 12000 rpm for 2 minutes.

The cosmetic composition of a foundation has satisfactory drying time, water resistance and color.

### Example 9: Preparation of a Cosmetic Hair Serum Composition

A cosmetic hair serum composition is prepared from a volatile mixture of alkanes consisting of 45% by mass of 2,3,6-trimethylheptane and 55% by mass of n-dodecane. This volatile mixture of alkanes is prepared by a process similar to that described in Example 1.

The detailed composition of this cosmetic hair serum formulation is given in Table 13 below:

Cosmetic composition of a hair serum

| Phase | Trade name | INCI | % by mass |
|---|---|---|---|
| 1 | BELSIL DL 1,000,000 | DIMETHICONE | 12 |
| | H110 from BRB | DIMETHICONOL | 5 |
| | VEGETABLE SQUALENE ST | OLEA EUROPAEA (OLIVE) | 6 |
| | BIOSYNTHIS | OIL UNSAPONIFIABLES | |
| | VEGELIGHT 1214 | COCONUT ALKANE | 19 |
| 2 | Volatile mixture of alkanes | | 55 |
| | (2,3,6-trimethylheptane/n-dodecane) | | |
| 3 | KARILEINE OIL BIOSYNTHIS | BUTYROSPERMUM PARKII | 3 |
| | | (SHEA) BUTTER | |

| | |
|---|---|
| TOTAL | 100 |

Phase 1 ingredients are dispersed in the Itra-Turrax™.

Phases 1, 2 and 3 are then mixed to obtain a hair serum.

### Example 10: Biodegradability of a Volatile Mixture of Alkanes Prepared by the Process According to the Invention

The biodegradability of a volatile mixture of alkanes consisting of 35% by mass of 2,3,6-trimethylheptane and 65% by mass of n-dodecane prepared by a process similar to that described in Example 1 was measured according to OECD method 301F.

The OECD 301F method is used to characterize the ease of biodegradation of a substance by a manometric respirometry test.

The volatile mixture of alkanes tested shows a biodegradability of 58% according to the OECD 301F method.

1-19. (canceled)

20. A process for preparing a volatile mixture of alkanes comprising the following steps:
step 1: producing a Guerbet alcohol by a Guerbet synthesis from a biosourced linear or branched alcohol with a mass purity greater than or equal to 80%,
step 2: adding to the Guerbet alcohol obtained during step 1 at least one biosourced linear or branched alcohol to form a mixture of biosourced alcohols,
step 3: obtaining a volatile mixture of alkanes consisting of the branched alkane from Guerbet alcohol and at least one alkane from the at least one biosourced alcohol added in step 2 by a dehydration reaction followed by a hydrogenation reaction of the mixture of biosourced alcohols formed in step 2.

21. The process according to claim 20, wherein the biosourced linear or branched alcohol has a mass purity greater than or equal to 80%.

22. The process according to claim 20, wherein step 1 is carried out from a linear or branched biosourced alcohol in C≤10.

23. The process according to claim 20, wherein step 1 is carried out using isoamyl alcohol.

24. The process according to claim 20, wherein the alcohol used in step 1 and the alcohol added to the Guerbet alcohol in step 2 are of plant origin.

25. The process according to claim 20, wherein the at least one alcohol added to the Guerbet alcohol during step 2 is a linear or branched biosourced C>10 alcohol.

26. The method according to claim 25, wherein the at least one linear or branched biosourced C>10 alcohol is a linear alcohol chosen from the group consisting of n-decanol, n-dodecanol and n-tetradecanol.

27. The process according to claim 20, wherein the volatile mixture of alkanes obtained in step 3 contains at least 20% by mass of the branched alkane derived from Guerbet alcohol and at least 20% by mass of at least one alkane derived from the at least one biosourced alcohol added in step 2, relative to the total mass of the volatile mixture of alkanes.

28. The process according to claim 20, wherein the volatile mixture of alkanes obtained in step 3 contains 20 to 80% by mass of 2,3,6-trimethylheptane and 20 to 80% by mass of at least one linear alkane chosen from the group consisting of n-decane, n-dodecane and n-tetradecane, relative to the total mass of the volatile mixture of alkanes.

29. The process according to claim 20, wherein the volatile mixture of alkanes obtained in step 3 contains 20 to 80% by mass of 2,3,6-trimethylheptane and 20 to 80% by mass of n-dodecane relative to the total mass of the volatile mixture of alkanes.

30. The process according to claim 20, wherein the volatile mixture of alkanes obtained in step 3 has a flash point below 55° C. according to standard ASTM D93.

31. The process according to claim 20, wherein the organic carbon present in the volatile mixture of alkanes obtained in step 3 has ratios ¹⁴C/¹²C or ¹⁴C/¹³ determined by radiocarbon analysis according to one of the following standards ASTM D6866, EN 16640 or EN 16785-1 which correspond to the ratios of living plant products.

32. The process according to claim 20, wherein the volatile mixture of alkanes obtained in step 3 has a biodegradability of at least 50% according to the OECD 301F method.

33. A process for preparing a cosmetic composition, comprising a step of mixing the volatile mixture of alkanes obtained by a process according to claim 20 with at least one compound chosen from non-volatile oils, glossy oils, pasty fatty substances, gelling agents, film-forming polymers, waxes, antioxidants, pigments, dyes, surfactants, an aqueous phase, and mixtures thereof.

34. The process for preparing a cosmetic composition according to claim 33, wherein the mass percentage of the volatile mixture of alkanes is greater than or equal to 5% relative to the total mass of the composition.

35. The process for preparing a cosmetic composition according to claim 33, wherein the cosmetic composition has a percentage greater than or equal to 80% of carbon 14 (14° C.) determined according to one of the following standards ASTM D6866, EN 16640 or EN 16785-1.

36. A method comprising preparing a cosmetic composition for lipstick, long-lasting lipstick, or lip gloss comprising the volatile mixture of alkanes obtained by the process according to claim 20.

37. A method comprising preparing a cosmetic eye makeup composition comprising the volatile mixture of alkanes obtained by the process according to claim 20.

38. A method comprising preparing a cosmetic composition applicable to the skin comprising the volatile mixture of alkanes obtained by the process according to claim 20.

39. A method comprising preparing a cosmetic composition for the treatment of keratin fibers comprising the volatile mixture of alkanes obtained by the process according to claim 20.
